(19) United States
(12) Patent Application Publication
(10) Pub. No.: US 2024/0052370 A1
(43) Pub. Date: Feb. 15, 2024

(54) MODULATING CELLULAR REPAIR MECHANISMS FOR GENOMIC EDITING

(71) Applicant: Inscripta, Inc., Pleasanton, CA (US)

(72) Inventors: Jacob BRAMMER, Pleasanton, CA (US); Jeffrey QUINN, Pleasanton, CA (US); Erik ZIMMERMAN, Pleasanton, CA (US)

(73) Assignee: Inscripta, Inc., Pleasanton, CA (US)

(21) Appl. No.: 18/359,382

(22) Filed: Jul. 26, 2023

Related U.S. Application Data
(60) Provisional application No. 63/369,601, filed on Jul. 27, 2022.

(51) Int. Cl. C12N 15/90; C12N 9/22; C12N 9/12; C12N 15/11; C12N 15/85

(52) U.S. Cl. CPC C12N 15/90; C12N 9/22; C12N 9/1276; C12N 15/11; C12N 15/85; C12N 2310/20; C12N 2800/107

(57) ABSTRACT

The present disclosure relates to methods and compositions of matter to increase the percentage of edited cells in a cell population when employing nucleic-acid guided editing methods, as well as systems and instruments for performing these methods and using these compositions.

### CROSS REFERENCED TO RELATED APPLICATIONS

This application claims benefit of U.S. Provisional Application No. 63/369,601, filed Jul. 27, 2022, which is incorporated by reference herein in their entirety.

### INCORPORATION OF ELECTRONIC SEQUENCE LISTING

A sequence listing conforming to the rules of WIPO Standard ST.26 is hereby incorporated by reference in its entirety. Said sequence listing has been filed as an electronic document via Patent Center encoded as XML in UTF-8 text. The electronic document is named “SL-P35295US01.xml”, and is 93,091 bytes in size (measured in MS-Windows®) and was created on Jul. 24, 2023.

### FIELD

This disclosure relates to methods and compositions of matter to increase the percentage of edited cells in a cell population when employing nucleic-acid guided editing methods, as well as systems and instruments, such as automated multi-module instruments, for performing these methods and using these compositions.

### BACKGROUND

In the following discussion, certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently, various nucleases have been identified that allow manipulation of gene sequence, and hence, gene function. The nucleases include nucleic acid-guided nucleases, such as RNA-guided nucleases, which enable researchers to generate permanent edits in live cells. Of course, it is desirable to attain the highest rates of precise edits possible in a cell population; however, in many instances, the percentage of precisely edited cells resulting from nucleic acid-guided nuclease editing can be relatively low, e.g., less than 1%. One of the many factors contributing to such low edit rates include native cellular repair mechanisms, such as DNA mismatch repair (MMR) systems, which may impede editing and produce undesirable byproducts.

There is thus a need in the art of nucleic acid-guided nuclease editing for improved methods, compositions, instruments, and systems for increasing the efficiency of editing. The present disclosure addresses this need.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In certain embodiments, a system for nucleic acid-guided editing in a genome of a cell is provided, the system comprising: a CREATE fusion gRNA (CFgRNA) or a polynucleotide sequence encoding a CFgRNA, the CFgRNA comprising an edit to a target genomic locus of a cell; a nickase-RT fusion enzyme or a polynucleotide sequence encoding the nickase-RT fusion enzyme; and a mismatch repair (MMR) perturbation agent, the MMR perturbation agent comprising a first MMR polypeptide or a polynucleotide sequence encoding the first MMR polypeptide, the first MMR polypeptide comprising a wild-type MMR polypeptide from the cell or another species.

In certain embodiments, a cell is provided, the cell comprising: a system for nucleic acid-guided editing in the genome of the cell, the system comprising: a CREATE fusion gRNA (CFgRNA) or a polynucleotide sequence encoding a CFgRNA, the CFgRNA comprising an edit to a target genomic locus of a cell; a nickase-RT fusion enzyme or a polynucleotide sequence encoding the nickase-RT fusion enzyme; and a mismatch repair (MMR) perturbation agent, the MMR perturbation agent comprising a first MMR polypeptide or a polynucleotide sequence encoding the first MMR polypeptide, the first MMR polypeptide comprising a wild-type MMR polypeptide from the cell or another species.

In certain embodiments, a method for performing nucleic acid-guided editing in a genome of a cell is provided, the method comprising: introducing into a cell a system for nucleic acid-guided editing in a genome of the cell, the system comprising: a CREATE fusion gRNA (CFgRNA) or a polynucleotide sequence encoding a CFgRNA, the CFgRNA comprising an edit to a target genomic locus of a cell; a nickase-RT fusion enzyme or a polynucleotide sequence encoding the nickase-RT fusion enzyme; and a mismatch repair (MMR) perturbation agent, the MMR perturbation agent comprising a first MMR polypeptide or a polynucleotide sequence encoding the first MMR polypeptide, the first MMR polypeptide comprising a wild-type MMR polypeptide from the cell or another species; and providing conditions to allow the CFgRNA and the nickase-RT fusion enzyme to bind to and edit the target genomic locus of the cell while at least the first MMR protein is expressed.

In certain embodiments, a system for nucleic acid-guided editing in a genome of a cell is provided, the system comprising: a CREATE fusion gRNA (CFgRNA) or a polynucleotide sequence encoding a CFgRNA, the CFgRNA comprising an edit to a target genomic locus of a cell; a nickase-RT fusion enzyme or a polynucleotide sequence encoding the nickase-RT fusion enzyme; and a mismatch repair (MMR) perturbation agent, the MMR perturbation agent comprising a first MMR polypeptide or a polynucleotide sequence encoding the first MMR polypeptide, the first MMR polypeptide comprising a wild-type MLH1 from the cell or another species, wherein the MMR perturbation agent further comprises a second MMR polypeptide or a polynucleotide sequence encoding the second MMR polypeptide, the second MMR polypeptide comprising a K675R variant of MSH2.

These aspects and other features and advantages of the disclosure are described below in more detail.

### BRIEF DESCRIPTION OF THE DRAWINGS

The foregoing and other features and advantages of the present disclosure will be more fully understood from the following detailed description of illustrative embodiments taken in conjunction with the accompanying drawings in which:

FIG. 1A is a simplified block diagram of an exemplary method for improved editing of live cells, wherein cellular mismatch repair (MMR) mechanisms of the cells are disrupted via introduction of an MMR perturbation agent during the editing process. FIG. 1B is a simplified graphic depiction of the mechanism of nucleic acid-guided nuclease editing, and more particularly, nucleic acid-guided nickase-reverse transcriptase fusion enzyme editing (“nickase-RT fusion editing”), according to certain embodiments described herein.

FIG. 2A schematically depicts an exemplary two-vector system for editing of live cells, the two vectors comprising a nuclease, an editing cassette, a selectable marker, and a component of an MMR perturbation agent. FIG. 2B schematically depicts an exemplary three-vector system for editing of live cells, wherein a first vector comprises a component of an MMR perturbation agent, a second vector comprises a nuclease, and a third vector comprises an editing cassette.

FIG. 3 schematically depicts an exemplary CF editing cassette for nickase-RT fusion editing of live cells.

FIGS. 4A-4D graphically illustrate the edit rates (% BFP+) in iPSCs for each of a plurality of GFP-to-BFP CFgRNAs as screened against several MMR nucleotide constructs found to facilitate improved editing performance as compared to baseline.

FIGS. 5A-5D illustrate another example of CF editing carried out in iPSCs with co-expression of exogenous MMR proteins (delivered as nucleotide constructs) along with CF editing machinery. FIG. 5A schematically depicts an exemplary two-vector system for editing of live cells, the two vectors comprising a CF enzyme, a CFgRNA, a selectable marker, and an MMR construct. FIG. 5B depicts a simplified graphic of a workflow for editing cells with the two-vector system of FIG. 5A. FIG. 5C graphically illustrates the precise edit rates (% correct intended edit) at each target genomic locus for experimental samples transfected with different MMR proteins. FIG. 5D graphically illustrates the precise edit rates (% correct intended edit) observed in iPSCs at various endogenous loci for a top performing MMR protein from FIG. 5C. FIG. 5E depicts the editing performance fold-improvement over baseline for the top performing MMR protein from FIG. 5C.

FIGS. 6A-6G illustrate another example of CF editing carried out in iPSCs to determine the effects of co-expressing a combination of MMR proteins along with CF editing machinery, as compared to individual MMR proteins or no MMR proteins. FIG. 6A graphically illustrates the on-target edit rates (correct intended “edit fraction”) for samples having one, two, or no MMR proteins delivered thereto, and FIG. 6B graphically depicts the average fold-improvement for experimental samples over baseline. FIG. 6C graphically depicts the occurrence of NHEJ (NHEJ fraction) for each of the above conditions as averaged across all genomic targets. FIG. 6D graphically illustrates the binned increase in editing with each MMR protein individually or in combination at target loci having different basal editing efficiencies. FIG. 6E graphically illustrates the average fold-improvement (vs control) with each MMR protein individually or in combination for different edit types tested (insertion, swap, or swap/insertion). FIG. 6F illustrates the average fold-improvement (vs control) with each MMR protein individually or in combination based on nick-to-edit distance of the tested CFgRNAs (1-3 bases, 4-6 bases, or 7+ bases). FIG. 6G illustrates the average fold-improvement (vs control) with each MMR protein individually or in combination based on edit length of the tested CFgRNAs (2, 3, 4, 5, or 6+ bp).

It should be understood that the drawings are not necessarily to scale, and that like reference numbers refer to like features.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Berg et al. (2002) Biochemistry, 5′ Ed., W.H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this disclosure belongs. All publications mentioned herein are incorporated by reference for the purpose of describing and disclosing devices, methods and cell populations that may be used in connection with the presently described disclosure.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

In the following description, numerous specific details are set forth to provide a more thorough understanding of the present disclosure. However, it will be apparent to one of ordinary skill in the art that the present disclosure may be practiced without one or more of these specific details. In other instances, well-known features and procedures well known to those skilled in the art have not been described in order to avoid obscuring the disclosure.

The terms “cellular DNA repair perturbation agent” or “DNA repair perturbation agent,” as used herein, refer to an agent, typically biological, for modulating, disrupting, or even inhibiting one or more DNA repair pathways of a cell. Congruently, a “DNA mismatch repair perturbation agent,” “mismatch repair perturbation agent,” or “MMR perturbation agent” refers to an agent for disrupting or inhibiting a DNA mismatch repair (MMR) pathway of a cell. In the context of the present disclosure, a DNA repair perturbation agent may refer to one or more polypeptides or proteins, or a nucleotide sequences encoding the polypeptides or proteins, that when expressed in a cell, disrupt or inhibit cellular DNA repair pathways. A DNA repair perturbation agent may comprise naturally occurring (e.g., wild-type) nucleotide sequences, polypeptides, or proteins of a cell, or non-naturally occurring (e.g., a variant or exogenous) nucleotide sequences, polypeptides, or proteins. In still further embodiments, a DNA repair perturbation agent or MMR perturbation agent may comprise a chemical agent.

The terms “cellular DNA repair protein,” “DNA repair protein,” “repair protein,” “cellular DNA repair polypeptide,” “DNA repair polypeptide,” and “repair polypeptide” as used herein refer to proteins and/or polypeptides which participate in cellular DNA repair mechanisms or pathways to repair “damaged” DNA during replication, recombination, and other processes.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The terms “CREATE fusion editing” or “CF editing” in the context of the current methods and compositions refers to an editing technique that uses a nuclease editing enzyme having nickase activity in conjunction with one or more nucleic acids to facilitate editing. In specific embodiments, CF editing methods utilize a fusion protein, such as a nucleic acid-guided nickase/reverse transcriptase fusion, and a nucleic acid encoding one or more editing gRNAs comprising a region complementary to a target region of a nucleic acid. The one or more gRNAs are covalently linked to a repair template comprising a region homologous to the target region and having a mutation, e.g., an edit, of at least one nucleotide. For information regarding CF editing see, e.g., U.S. Pat. Nos. 10,689,669; 11,268,078; 11,268,088; U.S. Ser. No. 16/740,421; and PCT Nos. PCT/US2020/023725 and PCT/US2019/048607.

The terms “CREATE fusion editing cassette” or “CF editing cassette” in the context of the current methods and compositions refers to a nucleic acid molecule comprising a coding sequence for transcription of a CREATE fusion gRNA or “CFgRNA” to effect editing in a nucleic acid-guided nickase/reverse transcriptase fusion system where the CFgRNA is designed to bind to and facilitate editing of one or both DNA strands in a target locus.

The terms “CREATE fusion editing components” or “CF editing components” refers to one or both of a nucleic acid-guided nickase enzyme/reverse transcriptase fusion protein (“nickase-RT fusion”) and a CREATE fusion editing cassette (“CF editing cassette”) and/or CREATE fusion gRNA (“CFgRNA”) to effect editing in live cells.

The terms “CREATE fusion gRNA” or “CFgRNA” refer to a single RNA molecule comprising two portions, the first portion being a gRNA and the second portion being a repair template covalently linked to the gRNA and comprising an edit to a target locus of a cell genome.

The term “editing cassette” refers to a nucleic acid molecule comprising a coding sequence for transcription of a guide nucleic acid or gRNA covalently linked to a coding sequence for transcription of a donor template.

The terms “guide nucleic acid” or “guide RNA” or “gRNA” refer to a polynucleotide comprising 1) a guide sequence (e.g., a “spacer” sequence) capable of hybridizing to a target genomic locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on a donor DNA with a certain degree of homology with a target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The terms “DNA mismatch repair protein,” “mismatch repair protein,” “MMR protein,” “DNA mismatch repair polypeptide,” “mismatch repair polypeptide,” and “MMR polypeptide” refer to DNA repair proteins and/or polypeptides which participate in cellular DNA mismatch repair (MMR) mechanisms. Exemplary MMR proteins include MSH2, MSH6, MLH1, and PMS2. For more information regarding MMR and MMR proteins, see, e.g., Li, GM. Mechanisms and functions of DNA mismatch repair. Cell Res 18: 85-98 (2008); Kolodner, R D., and Marsischky, GT. Eukaryotic DNA mismatch repair. Current Opinion in Genetics & Development 9(1): 89-96 (1999); Kunkel, T A, and Erie, DA. DNA Mismatch Repair. Annual Review of Biochemistry 74:681-710 (2005); Friedhoff et al. Protein-protein interactions in DNA mismatch repair. DNA Repair 38: 50-57 (2016); all of which are herein incorporated in their entirety by reference for all purposes.

The term “nucleic acid” or “polynucleotide” refers to deoxyribonucleic acids (DNA) or ribonucleic acids (RNA) and polymers thereof in either single- or double-stranded form. Unless otherwise indicated, the terms encompass nucleic acids containing known analogues or natural nucleotides that have similar binding properties as the reference nucleic acid and are metabolized in a manner similar to naturally occurring nucleotides. Unless otherwise indicated, in addition to the sequence specifically stated, a particular nucleic acid sequence also implicitly encompasses conservatively modified variants thereof (e.g., degenerate codon substitutions), alleles, orthologues, SNPs, and complementary sequences. The term nucleic acid is used interchangeably with DNA, RNA, cDNA, gene, and mRNA encoded by a gene.

As used herein, “nucleic acid-guided nickase/reverse transcriptase fusion” or “nickase-RT fusion” refers to a nucleic acid-guided nickase—or nucleic acid-guided nuclease or CRISPR nuclease that has been engineered to act as a nickase rather than a nuclease that initiates double-stranded DNA breaks—where the nucleic acid-guided nickase is fused to a reverse transcriptase, which is an enzyme used to generate cDNA from an RNA template. In certain embodiments, “nucleic acid-guided nickase/reverse transcriptase fusion” or “nickase-RT fusion” refers to two or more nucleic acid-guided nickases—or nucleic acid-guided nucleases or CRISPR nucleases that have been engineered to act as nickases rather than nucleases that initiate double-stranded DNA breaks—where the nucleic acid-guided nickases are fused to a reverse transcriptase. For information regarding nickase-RT fusions see, e.g., U.S. Pat. No. 10,689,669 and U.S. Ser. No. 16/740,421.

“Nucleic acid-guided editing components” refers to one or both of a nickase or nuclease and a guide nucleic acid or editing cassette.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

As used herein, the terms “protein,” “peptide,” and “polypeptide” are used interchangeably herein and refer to a polymer of amino acid residues. Proteins may or may not be made up entirely of amino acids transcribed by any class of any RNA polymerase I, II or III.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible.

As used herein, the term “repair template,” in the context of a CREATE fusion editing system employing a nickase-RT fusion enzyme refers to a nucleic acid that is designed to serve as a template (including a desired edit) to be incorporated into target DNA via reverse transcriptase.

As used herein, the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target genomic DNA locus”, “target locus”, or “target genomic locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The term “variant” may refer to a polypeptide or polynucleotide that differs from a reference polypeptide or polynucleotide (e.g., a wild-type) but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In the present disclosure, a single vector may include a coding sequence for a nickase-RT fusion enzyme and a CF editing cassette and/or CFgRNA sequence to be transcribed. In other embodiments, however, two or more vectors e.g., an engine vector comprising the coding sequence for the nickase-RT fusion enzyme, and an editing vector, comprising the CFgRNA sequence to be transcribed may be used.

The present disclosure relates to methods, compositions, instruments, and systems for improved nucleic acid-guided nuclease editing. With the present compositions and methods, genomic editing efficiency is improved via modulation or disruption of cellular DNA repair mechanisms, such as DNA mismatch repair (MMR) mechanisms. Without being bound to any scientific theory, MMR can impede genomic editing by reversion of the intended edit back to the original, unedited sequence, and/or by promotion of undesired indel byproducts. More particularly, certain embodiments of the present disclosure facilitate improved editing efficiency via utilization of proteins and/or polypeptides involved in or affecting cellular repair mechanisms, or polynucleotides encoding such proteins and/or polypeptides, which may be co-delivered into and/or co-expressed in desired cells along with editing machinery (e.g., an editing gRNA covalently linked to an intended edit and a corresponding nucleic acid-guided nuclease or nickase). The overexpression, or upregulation, of such proteins and/or polypeptides perturbs the native repair mechanisms of the cells, leading to improved editing outcomes.

Thus, in some aspects, there is provided a system for nucleic acid-guided editing in a genome of a cell, comprising: a CREATE fusion gRNA (CFgRNA) or a polynucleotide sequence encoding a CFgRNA, the CFgRNA comprising an edit to a target genomic locus of a cell; a nickase-RT fusion enzyme or a polynucleotide sequence encoding the nickase-RT fusion enzyme; and a mismatch repair (MMR) perturbation agent, the MMR perturbation agent comprising a first MMR polypeptide or a polynucleotide sequence encoding the first MMR polypeptide, the first MMR polypeptide comprising a wild-type MMR polypeptide.

In some aspects, the first MMR polypeptide comprises an MMR protein or a portion thereof. In some aspects, the first MMR polypeptide comprises an MMR protein complex comprising two or more wild-type MMR proteins or portions thereof (e.g., a dimer). Examples of suitable MMR proteins include MLH1, MLH1co, MLH2, MLH3, MSH2, MSH3, MSH6, PMS1, PMS2, etc. Examples of suitable MMR protein complexes include MutL (MutLα, MutLβ, MutLγ), MutS (MutSα, MutSβ), etc., which may comprises one or more of MLH1, MLH2, MLH3, MSH2, MSH3, MSH6, PMS1, PMS2, etc.

In some aspects, the first MMR polypeptide comprises wild-type MSH2 or portion thereof, and has an amino acid sequence of SEQ ID NO: 1, SEQ ID NO: 2, or SEQ ID NO: 3.

In some aspects, the first MMR polypeptide comprises wild-type MLH1 or portion thereof, and has an amino acid sequence of SEQ ID NO: 4, SEQ ID NO: 5, or SEQ ID NO: 6.

In some aspects, the first MMR polypeptide comprises wild-type MSH6 or portion thereof, and has an amino acid sequence of SEQ ID NO: 7, SEQ ID NO: 8, or SEQ ID NO: 9.

In some aspects, the first MMR polypeptide comprises wild-type PMS2 or portion thereof, and has an amino acid sequence of SEQ ID NO: 10, SEQ ID NO: 11 or SEQ ID NO: 12.

In some aspects, the first MMR polypeptide comprises wild-type MLH2 or portion thereof, and has an amino acid sequence of SEQ ID NO: 49.

In some aspects, the first MMR polypeptide comprises wild-type MLH3 or portion thereof, and has an amino acid sequence of SEQ ID NO: 50.

In some aspects, the first MMR polypeptide comprises wild-type MSH3 or portion thereof, and has an amino acid sequence of SEQ ID NO: 51.

In some aspects, the first MMR polypeptide comprises wild-type PMS1 or portion thereof, and has an amino acid sequence of SEQ ID NO: 52.

In some aspects, the first MMR polypeptide is a eukaryotic MMR polypeptide, such as a mammalian MMR polypeptide. In specific aspects, the first MMR polypeptide is a human MMR polypeptide. In some aspects, the first MMR polypeptide is a prokaryotic MMR polypeptide, such as a bacterial MMR polypeptide. In specific aspects, the first MMR polypeptide is an E. coli MMR polypeptide. In some aspects, the first MMR polypeptide is an archaeal MMR polypeptide.

In some aspects, MMR perturbation agent further comprises a second MMR polypeptide or a polynucleotide sequence encoding the second MMR polypeptide, the second MMR polypeptide comprising an MMR polypeptide variant, e.g., a dominant-negative variant that is catalytically impaired.

In some aspects, the second MMR polypeptide comprises an MMR protein or a portion thereof. In some aspects, the second MMR polypeptide comprises an MMR protein complex comprising two or more MMR proteins or portions thereof (e.g., a dimer, trimer, etc.). Examples of suitable MMR proteins include MLH1, MLH1co, MLH2, MLH3, MSH2, MSH3, MSH6, PMS1, PMS2, etc. Examples of suitable MMR complexes include MutL (MutLα, MutLβ, MutLγ), MutS (MutSα, MutSβ), etc., which may comprises one or more of MLH1, MLH2, MLH3, MSH2, MSH3, MSH6, PMS1, PMS2, etc.

In some aspects, the second MMR polypeptide comprises a point mutation variant of MSH2 or portion thereof, and has an amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21. In some aspects, the second MMR polypeptide comprises a domain truncation variant of MSH2 or portion thereof, and has an amino acid sequence of SEQ ID NO: 22, SEQ ID NO: 23, or SEQ ID NO: 24.

In some aspects, the second MMR polypeptide comprises a domain truncation variant of MLH1 or portion thereof, and has an amino acid sequence of SEQ ID NO: 25, SEQ ID NO: 26, or SEQ ID NO: 27. In some aspects, the second MMR polypeptide comprises a codon-optimized domain truncation variant of MLH1 or portion thereof, and has an amino acid sequence of SEQ ID NO: 43, SEQ ID NO: 44, or SEQ ID NO: 45. In some aspects, the second MMR polypeptide comprises a short deletion variant of MLH1 or portion thereof, and has an amino acid sequence of SEQ ID NO: 46, SEQ ID NO: 47, or SEQ ID NO: 48.

In some aspects, the second MMR polypeptide comprises a point mutation variant of MSH6 or portion thereof, and has an amino acid sequence of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36. In some aspects, the second MMR polypeptide comprises a domain truncation variant of MSH6 or portion thereof, and has an amino acid sequence of SEQ ID NO: 38, SEQ ID NO: 38, or SEQ ID NO: 39.

In some aspects, the second MMR polypeptide comprises a domain truncation variant of PMS2 or portion thereof, and has an amino acid sequence of SEQ ID NO: 40, SEQ ID NO: 41, or SEQ ID NO: 42.

In some aspects, the second MMR polypeptide comprises a ΔCTD (C-terminal domain) variant or ΔNTD (N-terminal domain) variant of an MMR protein.

In some aspects, the second MMR polypeptide comprises a K675R variant of MSH2. In some aspects, the second MMR polypeptide comprises a M688R variant of MSH2. In some aspects, the second MMR polypeptide comprises a ΔCTD variant of MSH2.

In some aspects, the second MMR polypeptide comprises a ΔNTD variant of MLH1. In some aspects, the second MMR polypeptide comprises a variant of MLH1 comprising a codon optimized NTD. In some aspects, the second MMR polypeptide comprises a Δ754-756 variant of MLH1.

In some aspects, the second MMR polypeptide comprises a F432A variant of MSH6. In some aspects, the second MMR polypeptide comprises a K11140R variant of MSH6. In some aspects, the second MMR polypeptide comprises a M1153R variant of MSH6. In some aspects, the second MMR polypeptide comprises a ΔCTD variant of MSH6.

In some aspects, the second MMR polypeptide comprises a ΔNTD variant of PMS2.

In some aspects, the second MMR polypeptide is a eukaryotic MMR polypeptide, such as a mammalian MMR polypeptide. In specific aspects, the second MMR polypeptide is a human MMR polypeptide. In some aspects, the second MMR polypeptide is a prokaryotic MMR polypeptide, such as a bacterial MMR polypeptide. In specific aspects, the second MMR polypeptide is an E. coli MMR polypeptide. In some aspects, the second MMR polypeptide is an archaeal MMR polypeptide.

In some aspects, the first and/or the second MMR polypeptide further comprise a nuclear localization site (NLS) at an N′ or C′ terminus thereof. In some aspects, the first and/or the second MMR polypeptide further comprise a promoter at an N′ or C′ terminus thereof.

In some aspects, the MMR perturbation agent further comprises a polypeptide comprising, or a polynucleotide sequence encoding, MEAF6, MPG, LIG1, LIG3 (A9), LIG3 (B5), FEN1, MRGBP, KAT7, RPA2, CHAF1B, ALKBHZ, NUDT1 (B4), BUDT1 (B8), portions thereof, and/or combinations thereof.

In further aspects, there is provided a system for nucleic acid-guided editing in a genome of a cell, comprising: a CREATE fusion gRNA (CFgRNA) or a polynucleotide sequence encoding a CFgRNA, the CFgRNA comprising an edit to a target genomic locus of a cell; a nickase-RT fusion enzyme or a polynucleotide sequence encoding the nickase-RT fusion enzyme; and a mismatch repair (MMR) perturbation agent, the MMR perturbation agent comprising a first polypeptide for perturbing an MMR mechanism or a polynucleotide sequence encoding the first polypeptide.

In some aspects, the first polypeptide comprises a wild-type polypeptide.

In some aspects, the first polypeptide comprises a variant or mutant polypeptide. In some aspects, the first polypeptide comprises a dominant-negative polypeptide variant.

In some aspects, the first polypeptide comprises an MMR protein or a portion thereof. In some aspects, the first polypeptide comprises an MMR protein complex comprising two or more MMR proteins or portions thereof (e.g., a dimer, trimer, etc.). Examples of suitable MMR proteins include MLH1, MLH1co, MLH2, MLH3, MSH2, MSH3, MSH6, PMS1, PMS2, etc. Examples of suitable MMR complexes include MutL (MutLα, MutLβ, MutLγ), MutS (MutSα, MutSβ), etc., which may comprises one or more of MLH1, MLH2, MLH3, MSH2, MSH3, MSH6, PMS1, PMS2, etc.

In some aspects, the first polypeptide comprises wild-type MSH2 or portion thereof, and has an amino acid sequence of SEQ ID NO: 1, SEQ ID NO: 2, or SEQ ID NO: 3. In some aspects, the first polypeptide comprises a point mutation variant of MSH2 or portion thereof, and has an amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 21. In some aspects, the first polypeptide comprises a domain truncation variant of MSH2 or portion thereof, and has an amino acid sequence of SEQ ID NO: 22, SEQ ID NO: 23, or SEQ ID NO: 24.

In some aspects, the first polypeptide comprises wild-type MLH1 or portion thereof, and has an amino acid sequence of SEQ ID NO: 4, SEQ ID NO: 5, or SEQ ID NO: 6. In some aspects, the first polypeptide comprises a domain truncation variant of MLH1 or portion thereof, and has an amino acid sequence of SEQ ID NO: 25, SEQ ID NO: 26, or SEQ ID NO: 27. In some aspects, the first polypeptide comprises a codon-optimized domain truncation variant of MLH1 or portion thereof, and has an amino acid sequence of SEQ ID NO: 43, SEQ ID NO: 44, or SEQ ID NO: 45. In some aspects, the first polypeptide comprises a short deletion variant of MLH1 or portion thereof, and has an amino acid sequence of SEQ ID NO: 46, SEQ ID NO: 47, or SEQ ID NO: 48.

In some aspects, the first polypeptide comprises wild-type MSH6 or portion thereof, and has an amino acid sequence of SEQ ID NO: 7, SEQ ID NO: 8, or SEQ ID NO: 9. In some aspects, the first polypeptide comprises a point mutation variant of MSH6 or portion thereof, and has an amino acid sequence of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36. In some aspects, the first polypeptide comprises a domain truncation variant of MSH6 or portion thereof, and has an amino acid sequence of SEQ ID NO: 38, SEQ ID NO: 38, or SEQ ID NO: 39.

In some aspects, the first polypeptide comprises wild-type PMS2 or portion thereof, and has an amino acid sequence of SEQ ID NO: 10, SEQ ID NO: 11, or SEQ ID NO: 12. In some aspects, the first polypeptide comprises a domain truncation variant of PMS2 or portion thereof, and has an amino acid sequence of SEQ ID NO: 40, SEQ ID NO: 41, or SEQ ID NO: 42.

In some aspects, the first polypeptide comprises wild-type MLH2 or portion thereof, and has an amino acid sequence of SEQ ID NO: 49.

In some aspects, the first polypeptide comprises wild-type MLH3 or portion thereof, and has an amino acid sequence of SEQ ID NO: 50.

In some aspects, the first polypeptide comprises wild-type MSH3 or portion thereof, and has an amino acid sequence of SEQ ID NO: 51.

In some aspects, the first polypeptide comprises wild-type PMS1 or portion thereof, and has an amino acid sequence of SEQ ID NO: 52.

In some aspects, the first polypeptide comprises MEAF6, MPG, LIG1, LIG3 (A9), LIG3 (B5), FEN1, MRGBP, KAT7, RPA2, CHAF1B, ALKBHZ, NUDT1 (B4), BUDT1 (B8), portions thereof, and/or combinations thereof.

In some aspects, the first polypeptide comprises a ΔCTD (C-terminal domain) variant or ΔNTD (N-terminal domain) variant of an MMR protein.

In some aspects, the first polypeptide comprises a K675R variant of MSH2. In some aspects, the first polypeptide comprises a M688R variant of MSH2.

In some aspects, the first polypeptide comprises a ΔNTD variant of MLH1. In some aspects, the first polypeptide comprises a variant of MLH1 comprising a codon optimized NTD. In some aspects, the first polypeptide comprises a Δ754-756 variant of MLH1.

In some aspects, the first polypeptide comprises a F432A variant of MSH6. In some aspects, the first polypeptide comprises a K11140R variant of MSH6. In some aspects, the first polypeptide comprises a M1153R variant of MSH6. In some aspects, the first polypeptide comprises a ΔCTD variant of MSH6.

In some aspects, the first polypeptide comprises a ΔNTD variant of PMS2.

In some aspects, the first polypeptide is a eukaryotic polypeptide, such as a mammalian polypeptide. In specific aspects, the first polypeptide is a human polypeptide. In some aspects, the first polypeptide is a prokaryotic polypeptide, such as a bacterial polypeptide. In specific aspects, the first polypeptide is an E. coli polypeptide. In some aspects, the first polypeptide is an archaeal polypeptide.

In some aspects, the first polypeptide comprises a MutS aptamer.

In some aspects, the MMR perturbation agent further comprises a second polypeptide or a polynucleotide sequence encoding the second polypeptide.

In some aspects, the second polypeptide comprises a wild-type polypeptide.

In some aspects, the second polypeptide comprises a variant or mutant polypeptide. In some aspects, the second polypeptide comprises a dominant-negative polypeptide variant.

In some aspects, the second polypeptide comprises an MMR protein or a portion thereof. In some aspects, the second polypeptide comprises an MMR protein complex comprising two or more MMR proteins or portions thereof (e.g., a dimer, trimer, etc.). Examples of suitable MMR proteins include MLH1, MLH1co, MLH2, MLH3, MSH2, MSH3, MSH6, PMS1, PMS2, etc. Examples of suitable MMR complexes include MutL (MutLα, MutLβ, MutLγ), MutS (MutSα, MutSβ), etc., which may comprises one or more of MLH1, MLH2, MLH3, MSH2, MSH3, MSH6, PMS1, PMS2, etc.

In some aspects, the second polypeptide comprises MEAF6, MPG, LIG1, LIG3 (A9), LIG3 (B5), FEN1, MRGBP, KAT7, RPA2, CHAF1B, ALKBHZ, NUDT1 (B4), BUDT1 (B8), portions thereof, and/or combinations thereof.

In some aspects, the second polypeptide comprises a ΔCTD (C-terminal domain) variant or ΔNTD (N-terminal domain) variant of an MMR protein.

In some aspects, the second polypeptide comprises a K675R variant of MSH2. In some aspects, the second polypeptide comprises a M688R variant of MSH2. In some aspects, the second polypeptide comprises a ΔCTD variant of MSH2.

In some aspects, the second polypeptide comprises a ΔNTD variant of MLH1. In some aspects, the second polypeptide comprises a variant of MLH1 comprising a codon optimized NTD. In some aspects, the second polypeptide comprises a Δ754-756 variant of MLH1.

In some aspects, the second polypeptide comprises a F432A variant of MSH6. In some aspects, the second polypeptide comprises a K11140R variant of MSH6. In some aspects, the second polypeptide comprises a M1153R variant of MSH6. In some aspects, the second polypeptide comprises a ΔCTD variant of MSH6.

In some aspects, the second polypeptide comprises a ΔNTD variant of PMS2.

In some aspects, the second polypeptide is a eukaryotic polypeptide, such as a mammalian polypeptide. In specific aspects, the second polypeptide is a human polypeptide. In some aspects, the second polypeptide is a prokaryotic polypeptide, such as a bacterial polypeptide. In specific aspects, the second polypeptide is an E. coli polypeptide. In some aspects, the second polypeptide is an archaeal polypeptide.

In some aspects, the second polypeptide comprises a MutS aptamer.

In some aspects, the first and/or the second polypeptide further comprise a nuclear localization site (NLS) at an N′ or C′ terminus thereof. In some aspects, the first and/or the second polypeptide further comprise a promoter at an N′ or C′ terminus thereof.

In some aspects, there is provided a system for nucleic acid-guided editing in a genome of a cell, the system comprising: a CREATE fusion gRNA (CFgRNA) or a polynucleotide sequence encoding a CFgRNA, the CFgRNA comprising an edit to a target genomic locus of a cell; a nickase-RT fusion enzyme or a polynucleotide sequence encoding the nickase-RT fusion enzyme; and a mismatch repair (MMR) perturbation agent, the MMR perturbation agent comprising a first MMR polypeptide or a polynucleotide sequence encoding the first MMR polypeptide, the first MMR polypeptide comprising a wild-type MLH1 from the cell or another species, wherein the MMR perturbation agent further comprises a second MMR polypeptide or a polynucleotide sequence encoding the second MMR polypeptide, the second MMR polypeptide comprising a K675R variant of MSH2.

In some aspects, there is provided a cell comprising the above-described systems. In some aspects, the cell is a live cell. In some aspects, the cell is an isolated cell.

In some aspects, the cell comprises a eukaryotic cell, such as a mammalian cell or fungal cell. In specific aspects, the cell comprises a human cell, such as a human T cell. In specific aspects, the cell includes a HeLa cell, HAP1 cell, HEK cell (e.g., HEK293T), K562 cell, and the like.

In some aspects, the cell comprises a prokaryotic cell, such as a bacterial cell. In specific aspects, the cell comprises an E. coli cell.

In some aspects, the cell comprises an archaeal cell.

In some aspects, the cell comprises a stem cell, such as a human stem cell. In specific aspects, the cell comprises an induced pluripotent stem cell (iPSC).

In some aspects, there is provided a method for performing nucleic acid-guided nuclease editing with the above-described systems. In some aspects, the method comprises providing a cell comprising a target genomic locus; providing the CREATE fusion gRNA (CFgRNA) or the polynucleotide sequence encoding the CFgRNA; providing the nickase-RT fusion enzyme or the polynucleotide sequence encoding the nickase-RT fusion enzyme; providing the mismatch repair (MMR) perturbation agent; delivering the CFgRNA, the nickase-RT fusion enzyme, and the MMR perturbation agent to the cell; providing conditions to allow the CFgRNA and the nickase-RT fusion enzyme to bind to and edit the target genomic locus of the cell while the first MMR polypeptide and/or the second MMR polypeptide are expressed.

In some aspects, the method comprises providing a cell comprising a target genomic locus; providing the CREATE fusion gRNA (CFgRNA) or the polynucleotide sequence encoding the CFgRNA; providing the nickase-RT fusion enzyme or the polynucleotide sequence encoding the nickase-RT fusion enzyme; providing the mismatch repair (MMR) perturbation agent; delivering the CFgRNA, the nickase-RT fusion enzyme, and the MMR perturbation agent to the cell; providing conditions to allow the CFgRNA and the nickase-RT fusion enzyme to bind to and edit the target genomic locus of the cell while the first polypeptide and/or the second polypeptide are expressed.

In some aspects, there is provided a method for performing nucleic acid-guided editing in a genome of a cell, the method comprising: introducing into a cell any of the systems described above; and providing conditions to allow the CFgRNA and the nickase-RT fusion enzyme to bind to and edit the target genomic locus of the cell.

In some aspects, the method achieves at least a 1.5, 2, 3, 4, 5, 6, 7, 8, 9, 10, 12, 15, 18, 20, or 25 fold increase in editing efficiency compared to a control editing system not containing one or both of the first and the second MMR polypeptides.

In some aspects, the fold increase is achieved in at least 20%, 30%, 40%, 50%, 60%, 70%, 80%, 90% or 95% target genomic loci tested.

In some aspects, the fold increase is achieved in target genomic loci with an editing efficiency lower than 2%, 1%, 0.5%, 0.3%, 0.2%, 0.1%, 0.05%, 0.02%, 0.01%, 0.005% when editing using the control editing system.

In further aspects, the MMR perturbation agent comprises a plurality of MMR polypeptides or polynucleotide sequences encoding the MMR polypeptides. In some aspects, the MMR perturbation agent comprises two or more MMR polypeptides or polynucleotide sequences encoding the MMR polypeptides. In some aspects, two or more of the MMR polypeptides comprise the same or different MMR polypeptides.

In some aspects, the MMR perturbation agent comprises a plurality of polypeptides or polynucleotide sequences encoding the polypeptides. In some aspects, the MMR perturbation agent comprises two or more polypeptides or polynucleotide sequences encoding the polypeptides. In some aspects, two or more of the MR polypeptides comprise the same or different polypeptides.

In some aspects, a polynucleotide sequence encoding an MMR or other polypeptide comprises an open reading frame (ORF) of the MMR or other polypeptide.

In some aspects, an MMR or other polypeptide, or a nucleotide sequence encoding the MMR or other polypeptide, comprises a portion of a protein. In some aspects, the portion of the protein comprises one or more domains of the protein. In some aspects, the portion of the protein comprises an active domain of the protein.

In some aspects, an MMR or other protein comprises a wild-type or variant (e.g., mutant) protein. In some aspects, a variant protein comprises a codon-optimized variant, a point mutant, a domain truncation variant, a short deletion variant, etc.

In specific aspects, an MMR protein comprises a wild-type or variant MLH1 protein, a wild-type or variant MLH3 protein, a wild-type or variant MSH2 protein, a wild-type or variant MSH6 protein, a wild-type or variant PMS1 protein, a wild-type or variant PMS2 protein, a wild-type or variant component of an MMR complex (e.g., a wild-type or variant component of the MutSα-MutLα MMR complex), or the like.

In some aspects, an MMR polypeptide is under the control of a constitutive or inducible promoter. In specific aspects, the MMR polypeptide is under the control of a different promoter than that of an editing gRNA (e.g., CFgRNA) and/or nuclease or nickase (e.g., nickase-RT fusion enzyme).

In some aspects, the CFgRNA is a component of an editing cassette, e.g., a CREATE fusion editing cassette (“CF editing cassette,” defined infra), for performing nucleic acid-guided nuclease editing. In some aspects, the CFgRNA is under the control of a promoter at, e.g., a 5′ end of the CF editing cassette.

In some aspects, the CFgRNA comprises from 5′ to 3′: 1) an editing gRNA having a region of complementarity to a sequence of a target locus in which an edit is to be integrated, the editing gRNA comprising: a guide or spacer region, and a scaffold region recognized by a corresponding nickase or nuclease; and 2) a repair template covalently linked to the editing gRNA and comprising from 5′ to 3′: an optional post-edit homology (PEH) region, an edit region comprising the edit, a nick-to-edit region, and a primer binding site (PBS) or region.

In some aspects, the components of an editing gRNA, a CFgRNA, an editing cassette, and/or a CF editing cassette are contiguous. In some aspects, an editing gRNA is agnostic to the order of a spacer region and a scaffold region. In some aspects, a CFgRNA is agnostic to the order of the editing gRNA and repair template. In some aspects, an editing cassette or CF editing cassette is agnostic to the order of components thereof.

In some aspects, a nick-to-edit region of a repair template is from 2-250 nucleotides in length, or from 5-150 nucleotides in length, or from 0-150 nucleotides in length. In some aspects, the nick-to-edit region of the repair template is up to 1,000 nucleotides in length, up to 3,000 nucleotides in length, or up to 10,000 nucleotides in length.

In some aspects, a post-edit homology region of a repair template is from 2-50 nucleotides in length, from 4-40 nucleotides in length, or from 5-25 nucleotides in length. In some aspects, the post-edit homology region of the repair template is from 2-500 nucleotides in length, from 2-250 nucleotides in length, or from 2-100 nucleotides in length.

In some aspects, a CFgRNA, an editing cassette, and/or CF editing cassette further comprises an edit (e.g., 1, 2, 3, 4, 5, or up to 10 edits) to immunize a target locus to prevent re-nicking or re-cutting thereof. As discussed herein, in some aspects, an edit to immunize a target locus to prevent re-nicking is one that alters the proto-spacer adjacent motif (or other element) such that subsequent binding at the target locus by the nucleic acid-guided nuclease or nickase is impaired or prevented.

In some aspects, an editing cassette or a CF editing cassette comprises an RNA G-quadruplex region at, e.g., a 3′ end of a repair template to stabilize the editing cassette or CF editing cassette and improve target nicking or cleavage efficiency without inducing off-target activity.

In some aspects, a CF editing cassette comprises an amplification priming site or subpool primer binding site at, e.g., a 3′ end thereof.

In some aspects, a CF editing cassette comprises a melting temperature booster region at, e.g., a 5′ end thereof, which is a short protective DNA buffer sequence.

In some aspects, a CF editing cassette comprises regions of homology to a vector for gap-repair insertion of the cassette into the vector, such as an editing vector or engine vector.

In some aspects, a region of complementarity between an editing gRNA (e.g., a guide or spacer region) of a CFgRNA and a target locus is from 4-120 nucleotides in length, or from 5-80 nucleotides in length, or from 6-60 nucleotides in length, e.g., from 0-10 nucleotides in length, 10-20 nucleotides in length, 20-50 nucleotides in length, or 50-100 nucleotides in length.

In some aspects, an edit is from 1-1,000 nucleotides in length, or from 1-750 nucleotides in length, or from 1-500 nucleotides in length, or from 1-150 nucleotides in length, e.g., from 1-10 nucleotides in length, 10-20 nucleotides in length, 20-50 nucleotides in length, 50-100 nucleotides, 100-250 nucleotides, 250-500 nucleotides, 500-750 nucleotides, or 500-1,000 or more nucleotides in length. In some aspects, the edit is from 1,000-10,000 or more nucleotides in length, or from 2,000-8,000 or more nucleotides in length, or from 4,000-6,000 or more nucleotides in length.

In some aspects, an edit region comprises one or more edits, or two or more edits, or three or more edits, or four or more edits, or five or more edits.

In some aspects, an edit comprises one or more base swaps in the target locus.

In some aspects, an edit comprises an insertion in the target locus.

In some aspects, an edit comprises an insertion of recombinase sites, protein degron tags, promoters, terminators, alternative-splice sites, CpG islands, etc.

In some aspects, an edit comprises a deletion in the target locus.

In some aspects, an edit comprises a deletion of from 1 to 750 or more nucleotides at a target locus. In some aspects, the edit comprises a deletion of from 1 to 10 nucleotides, from 10 to 20 nucleotides, from 20 to 50 nucleotides, from 50 to 100 nucleotides, from 100 to 200 nucleotides, from 200 to 500 nucleotides or from 250 to 750 or more nucleotides at a target locus.

In some aspects, an edit comprises a deletion of introns, exons, repetitive elements, promoters, terminators, insulators, CpG islands, non-coding elements, retrotransposons, etc.

In some aspects, an edit comprises several types of edits and/or comprises more than one of one or more types of edits. For example, in some aspects, the edit comprises two or more base swaps (e.g., 2, 3, 4, 5, or from 1 to 20 or more base swaps), some or all of which can be adjacent to each other or nonadjacent to each other. In some aspects, the edit comprises one or more base swaps (e.g., 2, 3, 4, 5, or from 1 to 20 or more base swaps) and an insertion of one or more nucleotides (e.g., 2, 3, 4, 5, or from 1 to 20 or more nucleotides). In some aspects, the edit comprises one or more base swaps (e.g., 2, 3, 4, 5, or from 1 to 20 or more base swaps) and a deletion of one or more nucleotides (e.g., 2, 3, 4, 5, or from 1 to 20 or more nucleotides).

In some aspects, an edit is targeted for and/or effected in a coding region of the target locus.

In some aspects, an edit is targeted for and/or effected in a noncoding region of the target locus.

In some aspects, a nickase or nuclease includes a MAD-series nickase, nuclease, or a variant (e.g., orthologue) thereof. In some aspects, the nickase or nuclease nuclease includes a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MAD-series nickase, nuclease, variant thereof, and/or combination thereof.

In some aspects, a nickase or nuclease includes a Cas9 (also known as Csn1 and Csx12), nickase, nuclease, or a variant thereof.

In some aspects, a nickase or nuclease includes C2c1, C2c2, C2c3, Cas1, Cas1B, Cas2, Cas3, Cas4, Cas5, Cas6, Cas7, Cas8, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, or similar nickase, nuclease, variant thereof, and/or combination thereof.

In some aspects, such as embodiments wherein a CFgRNA and/or CF editing cassette is utilized, a nickase or nuclease is part of a fusion protein—i.e., a nucleic acid-guided nickase/reverse transcriptase fusion enzyme (a “nickase-RT fusion”)—that retains certain characteristics of nucleic acid-directed nickases or nuclease (e.g., the binding specificity and ability to cleave one or more DNA strands in a targeted manner) combined with another enzymatic activity, namely, reverse transcriptase activity. The reverse transcriptase portion of the nickase-RT fusion may use a CFgRNA to synthesize and edit at a “flap” created by the nickase portion on one or both DNA strands of a target locus, thereby circumventing some endogenous mismatch repair systems to integrate an edit.

In some aspects, a mismatch repair (MMR) perturbation agent (or components thereof), an editing gRNA, and a nickase or nuclease are introduced into a cell on a single vector (e.g., a single-part system). In certain embodiments, the MMR perturbation agent (or one or more components thereof), the editing gRNA, and the nickase or nuclease are introduced into the cell as a multi-part system, wherein the MMR perturbation agent (or component(s) thereof) may be introduced separately from the editing gRNA and/or the nickase or nuclease. For example, the MMR perturbation agent (or component(s) thereof) may be comprised on a first vector, and the editing gRNA and/or the nickase or nuclease may be comprised on a second vector (and/or third vector) co-delivered with the first vector.

In some aspects, the nickase or nuclease is introduced into the cells as a DNA molecule coding for the nickase or nuclease separately or linked to the mismatch repair MMR perturbation agent (or component(s) thereof) and/or the editing gRNA, or the nickase or nuclease may introduced separately in polypeptide or protein form, or as part of a complex.

In some aspects, the MMR perturbation agent (or component(s) thereof), the editing gRNA, and/or the nucleic acid-guided nickase or nuclease are introduced into the cells on a linear or circular plasmid. In some aspects, the MMR perturbation agent (or component(s) thereof), the editing gRNA or editing cassette, and/or the nickase or nuclease are under the control of a constitutive or inducible promoter at, e.g., a 5′ end thereof. In some aspects, the MMR perturbation agent (or component(s) thereof) is under the control of a separate, independent (e.g., a different) promoter from the editing gRNA or editing cassette and/or the nickase or nuclease.

In some aspects, a vector comprising the MMR perturbation agent (or component(s) thereof), the editing gRNA, and/or the nucleic acid-guided nickase or nuclease further comprises an origin of replication and a selectable marker component, e.g., an antibiotic resistance gene or a fluorescent protein gene, for selection or enrichment of cells that have been transformed and/or edited. The selectable marker may be utilized for selective enrichment of transformed and/or edited cells. In some aspects, the selectable marker comprises an antibiotic resistance gene or a fluorescent protein.

In some aspects, there is provided a library of vector or plasmid backbones and/or a library of editing gRNAs or editing cassettes, and/or a library of CFgRNAs or CF editing cassettes, to be transformed into cells. In some aspects, the utilization of a library of gRNAs, cassettes, and/or a library of vector or plasmid backbones enables combinatorial or multiplex editing in the cells. A library of gRNAs, cassettes, or vectors may comprise gRNAs, cassettes, or vectors that have any combination of common elements and non-common or different elements as compared to other gRNAs, cassettes, or vectors within the pool. For example, a library of CF editing cassettes can comprise common priming sites or common nick-to-edit or post-edit homology regions, while also containing non-common or unique edits. Combinations of common and non-common elements are advantageous for multiplexing or combinatorial techniques disclosed herein.

In some aspects, a library of cassettes comprises at least 2 cassettes, or at least 10 cassettes, or at least 100 cassettes, or at least 1,000 cassettes, or at least 10,000 cassettes, or at least 100,000 cassettes, or at least 1,000,000 cassettes. In some aspects, a library of cassettes comprises from 5 to a 1,000,000 cassettes, or from 100 to 500,000 cassettes, or from 1,000 to 100,000 cassettes, or from 1,000 to 10,000 cassettes, or from 10,000 to 50,000 cassettes.

In some aspects, one or more cassettes in a library of editing cassettes or CF editing cassettes each comprise a different editing gRNA targeting a different target locus within the cell genome. In some aspects, one or more cassettes in a library of editing cassettes or CF editing cassettes each comprise a different edit to be incorporated within the cell genome.

In some aspects, there is provided a gRNA, an editing cassette, a CFgRNA, or a CF editing cassette comprising a barcode or other unique molecular identifier (UMI) for tracking of editing events. In some aspects, there is provided a trackable library comprising a plurality of gRNAs, cassettes, or a plurality or vectors comprising cassettes as disclosed herein. In some aspects, within the trackable library are distinct gRNA or cassette and barcode/UMI combinations, which when sequenced upon editing, facilitate tracking of editing events in a population of cells. Accordingly, when edits and barcodes are incorporated into a target genome, the incorporation of an edit is determined based on sequenced the barcode.

In some aspects, there is provided a gene-wide or genome-wide library of gRNAs, cassettes, or vectors comprising gRNAs or cassettes as disclosed herein.

In some aspects, there are provided methods of recursive or iterative rounds of editing operations. In some aspects, methods disclosed herein comprise 2 or more rounds of editing, such as 5 or more rounds of editing, such as 10 or more rounds of editing.

In some aspects, one or more unique barcodes or UMIs can be inserted in each round of multiple iterative or recursive editing operations.

In selected embodiments, the present disclosure provides compositions, systems, and methods suitable for utilization with automated modules, instruments, and systems for automated multi-module cell processing and nucleic acid-guided genome editing in cells. Automated systems for cell processing and editing that may be used can be found, e.g., in U.S. Pat. Nos. 10,253,316; 10,329,559; 10,323,242; 10,421,959; 10,465,185; 10,519,437; 10,584,333; 10,584,334; 10,647,982; 10,689,645; 10,738,301; and 10,738,663, which are incorporated by referenced herein in their entirety. In some aspects, the automated multi-module cell processing and editing instruments are designed for recursive genome editing, e.g., sequentially introducing multiple edits into genomes of one or more cells of a cell population through two or more editing operations within the instruments.

In some aspects, the methods, compositions, instruments, and systems described herein take advantage of cellular DNA repair mechanisms other than MMR to improve editing outcomes. For example, in such aspects, cellular DNA repair polypeptides and/or proteins other than those associated with MMR are expressed/overexpressed (e.g., upregulated) to perturb associated cellular repair pathways/mechanisms. Thus, in some aspects, there is provided a system for nucleic acid-guided editing in a genome of a cell, comprising: an editing gRNA or a polynucleotide sequence encoding an editing gRNA; a nickase or nuclease or a polynucleotide sequence encoding the nickase or nuclease; and a cellular DNA repair perturbation agent, the cellular DNA repair perturbation agent comprising a cellular DNA repair polypeptide or a polynucleotide sequence encoding the cellular DNA repair polypeptide.

In some aspects, there is provided a cell comprising the above-described system. In further aspects, there is provided a method for performing nucleic acid-guided nuclease editing with the above-described system.

In some aspects, the cellular DNA repair polypeptide comprises a polypeptide or protein involved in base excision repair (BER), nucleotide excision repair (NER), homologous recombination (HR), non-homologous end joining (NHEJ), other double-strand break response mechanism, or the like.

In some aspects, rather than co-delivering an MMR or cellular repair polypeptide or a polynucleotide sequence encoding the polypeptide along with editing machinery configured to effect an intended edit in a genome of a cell, the editing machinery further comprises a second edit to upregulate the expression of an MMR protein or DNA repair protein already encoded or expressed by the cell. Thus, in some aspects, there is provided a method for performing nucleic acid-guided nuclease editing in a genome of live cells, comprising: introducing a nucleic acid-guided nuclease, a first editing gRNA configured to guide the nucleic acid-guided nuclease to a first target locus of the live cells for introduction of a first edit to upregulate expression of a DNA repair protein, a first repair template comprising the first, a second editing gRNA configured to guide the nucleic acid-guided nuclease to a second target locus of the live cells for introduction of a second edit, a second repair template comprising the second edit; providing conditions to allow the nucleic acid-guided nuclease and the first editing gRNA to bind to and edit the first target locus of the cells and upregulate expression of the DNA repair protein; and, providing conditions to allow the nucleic acid-guided nuclease and the second editing gRNA to bind to and edit the second target locus of the cells with the second edit.

In still further aspects, reduced expression (e.g., knockdown) or knockout of cellular repair proteins during editing may perturb the native repair mechanisms of the cells, leading to improved editing outcomes. Thus, in some aspects, there is provided a method for performing nucleic acid-guided nuclease editing in a genome of live cells, comprising: introducing a nucleic acid-guided nuclease, a first editing gRNA comprising an intended library edit, and a second editing gRNA comprising a knockdown or knockout of a cellular repair protein; and providing conditions to allow the nucleic acid-guided nuclease and the first and second editing gRNAs to bind to and edit target loci of the cells, wherein editing with the second editing gRNA causes reduced expression of the cellular repair protein in the cell.

In some aspects, there is provided a method for performing nucleic acid-guided nuclease editing in a genome of live cells, comprising: introducing a nucleic acid-guided nuclease, an editing gRNA, and an exogenous nucleic acid into the cells, the exogenous nucleic acid comprising siRNA configured to reduce expression or a cellular repair protein of the cells; providing conditions to allow the nucleic acid-guided nuclease and the editing gRNA to bind to and edit a target locus of the cells; and, providing conditions for reduced expression of the cellular repair protein.

### Nucleic Acid-Guided Nuclease Editing, Generally

Certain embodiments described herein utilize nucleic acid-guided nuclease editing (i.e., RNA-guided nuclease or CRISPR editing) for performing genomic editing in live cells, e.g., for performing the competitive editing assay described above, to screen for plasmid backbones. In some embodiments, one or more edits are introduced in a single round of editing utilizing a plurality, e.g., a library, of candidate plasmid backbones.

In CRISPR-type editing generally, a nucleic acid-guided nuclease (or nickase) or CREATE fusion enzyme (“CF enzyme”) complexed with an appropriate synthetic guide nucleic acid (e.g., a gRNA or CFgRNA) in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease.

In general, a guide nucleic acid complexes with a compatible nucleic acid-guided nuclease or CF enzyme and can then hybridize with a target sequence, thereby directing the nuclease to the target sequence. A guide nucleic acid can comprise DNA or RNA; alternatively, a guide nucleic acid may comprise both DNA and RNA. In some embodiments, a guide nucleic acid may comprise modified or non-naturally occurring nucleotides. In certain embodiments, a guide nucleic acid may be encoded by a DNA sequence on a plasmid, or the coding sequence may and preferably does reside within an editing cassette assembled into a plasmid backbone. Methods and compositions for designing and synthesizing editing cassettes and libraries of editing cassettes are described in U.S. Pat. Nos. 10,240,167; 10,266,849; 9,982,278; 10,351,877; 10,364,442; 10,435,715; 10,465,207; 10,669,559; 10,711,284; 10,731,180; and 11,078,498; all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably, the guide sequence is 10-30 or 15-20 nucleotides long, or 15, 16, 17, 18, 19, or 20 nucleotides in length.

In certain embodiments of the present methods and compositions, the guide nucleic acids are provided as mRNAs or as sequences to be expressed from a candidate plasmid (or vector), and/or as sequences to be expressed from a cassette optionally inserted into a plasmid backbone, and comprise both a guide sequence and a scaffold sequence as a single transcript under the control of a promoter, e.g., an inducible or constitutive promoter. In certain embodiments, the guide nucleic acid may be part of an editing cassette that encodes a repair template for effecting an edit in the cellular target sequence, and/or one or more homology arms. Alternatively, the guide nucleic acid may not be part of the editing cassette and instead may be encoded on the plasmid backbone. For example, a sequence coding for a guide nucleic acid can be assembled or inserted into a plasmid backbone first, followed by insertion of the repair template in, e.g., an editing cassette. In other cases, the repair template in, e.g., an editing cassette can be inserted or assembled into a plasmid backbone first, followed by insertion of the sequence coding for the guide nucleic acid. In certain embodiments, the sequence encoding the guide nucleic acid and repair template are located together in a rationally designed editing cassette and are simultaneously inserted or assembled via gap repair into a plasmid backbone to create an editing plasmid (i.e., an editing vector).

The guide nucleic acids are engineered to target a desired target sequence (e.g., a cellular “editing” target sequence) by altering the guide sequence so that the guide sequence is complementary to a desired target sequence, thereby allowing hybridization between the guide sequence and the target sequence. The target sequence can be any polynucleotide endogenous or exogenous to a cell, e.g., a prokaryotic or eukaryotic cell, or in vitro. For example, the target sequence can be a polynucleotide residing in the nucleus of a eukaryotic cell. A target sequence can be a sequence encoding a gene product (e.g., a protein), a non-coding sequence (e.g., a regulatory polynucleotide, an intron, a proto-spacer adjacent motif (PAM) sequence, or “junk” DNA), or other sequence.

In general, to generate an edit in the target sequence, a guide nucleic acid/nuclease complex binds to the target sequence as determined by the guide RNA, and the nuclease or CF enzyme recognizes a PAM sequence adjacent to or in proximity to the target sequence. The precise preferred PAM sequence and length requirements for different nucleic acid-guided nucleases vary; however, PAMs typically are 2-10 or so base pairs in length and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In certain embodiments, genome editing of a cellular target sequence both introduces a desired DNA change to a cellular target sequence (an “intended” edit), e.g., the genomic DNA of a cell, and removes, mutates, or renders inactive a PAM region in the cellular target sequence (an “immunizing edit”), thereby rendering the target site immune to further nuclease binding. Rendering the PAM at the cellular target sequence inactive precludes additional editing of the cell genome at that cellular target sequence. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event may be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease or CF enzyme component of the editing system, a polynucleotide sequence encoding the nucleic acid-guided nuclease or CF enzyme can be codon optimized for expression in particular cell types, such as bacterial, yeast, and, here, mammalian cells. The choice of the nucleic acid-guided nuclease or CF enzyme to be employed depends on many factors, such as what type of edit is to be made in the target sequence and whether an appropriate PAM is located close to the desired target sequence. Nucleases and nickases of use in the methods described herein include but are not limited to Cas9, Cas12, MAD2, or MAD7, MAD2007 or other MADzymes and MADzyme systems (see U.S. Pat. Nos. 10,604,746; 10,655,114; 10,649,754; 10,876,102; 10,833,077; 11,053,485; 10,704,022; 10,745,678; 10,724,021; 10,767,169; 10,870,761; 10,011,849; 10,435,714; 10,626,416; 9,982,279; and 10,337,028; and U.S. Ser. Nos. 16/953,253; 17/374,628; 17,200,074; 17,200,089; 17/200,110; 16/953,233; 17/463,498; 63/134,938; 16/819,896; 17/179,193; and 16/421,783 for sequences and other details related to engineered and naturally-occurring MADzymes).

CF enzymes typically comprise a CRISPR nucleic acid-guided nuclease engineered to cut one DNA strand in the target DNA rather than making a double-stranded cut, and the nickase portion is fused to a reverse transcriptase. In specific aspects, the one or more nickases include MAD7 nickase, MAD2001 nickase, MAD2007 nickase, MAD2008 nickase, MAD2009 nickase, MAD2011 nickase, MAD2017 nickase, MAD2019 nickase, MAD297 nickase, MAD298 nickase, MAD299 nickase, or other MAD-series nickases, variants thereof, and/or combinations thereof as described in U.S. Pat. Nos. 10,883,077; 11,053,485; 11,085,030; 11,200,089; 11,193,115; and U.S. Ser. No. 17/463,498. A coding sequence for a desired nuclease or CF enzyme may be on an “engine vector” along with other desired sequences such as a selective marker(s), or a coding sequence for the desired nuclease may reside on an editing plasmid, or may be transfected into a cell as a protein.

In certain embodiments, nucleic acid-guided nucleases or CF enzymes comprise one or more nuclear localization sequences (NLSs), such as about or more than about 1, 2, 3, 4, 5, 6, 7, 8, 9, 10, or more NLSs, particularly as an element of the nuclease sequence. In some embodiments, an engineered nuclease comprises NLSs at or near the amino-terminus, NLSs at or near the carboxy-terminus, or a combination.

Another component of the editing system is the repair template comprising homology to the cellular target sequence. The repair template typically is designed to serve as a template for homologous recombination with a cellular target sequence cleaved by the nucleic acid-guided nuclease, or the repair template serves as the template for template-directed repair via the CF enzyme, as a part of the guide nucleic acid/nuclease complex. For the present methods and compositions, the repair template is typically on the same vector as, in the same editing cassette as, and/or part of the guide nucleic acid (e.g., a CFgRNA) for editing, and may be under the control of the same promoter as the guide nucleic acid (that is, a single promoter driving the transcription of both a gRNA and the repair template). A repair template polynucleotide may be of any suitable length, such as about or more than about 20, 25, 50, 75, 100, or more nucleotides in length. In certain preferred aspects, the repair template can be provided as an oligonucleotide of between 20-100 nucleotides, such as between 30-75 nucleotides. When optimally aligned, the repair template overlaps with (is complementary to) the cellular target sequence by, e.g., about 20, 25, 30, 35, 40, 50, 60, 70, 80, 90 or more nucleotides.

The repair template generally comprises two regions that are complementary to a portion of the cellular target sequence (e.g., homology arms). In certain embodiments of the present methods and compositions, the two homology arms flank an intended edit, e.g., at least one alteration as compared to the cellular target sequence, such as a DNA sequence insertion, which may be part of the repair template. In certain embodiments, the repair template comprises two homology arms that do not flank the intended edit. In such embodiments, the homology arms may be encoded on a plasmid backbone, or in an editing cassette with the edit.

Inducible editing is advantageous in that cells can be grown for several to many cell doublings before editing is initiated, which increases the likelihood that cells with edits will survive, as the double-strand cuts caused by active editing are largely toxic to the cells. This toxicity results both in cell death in the edited colonies, as well as possibly a lag in growth for the edited cells that do survive but must repair and recover following editing. However, once the edited cells have a chance to recover, the size of the colonies of the edited cells will eventually catch up to the size of the colonies of unedited cells. It is this toxicity, however, that is exploited herein to perform curing.

An editing cassette comprising a guide nucleic acid and repair template may further comprise one or more primer binding sites. The primer binding sites are used to amplify the editing cassette by using oligonucleotide primers as described infra and may be biotinylated or otherwise labeled. In addition, or alternative to the edit, an editing cassette may comprise a barcode. A barcode is a unique DNA sequence that corresponds to the repair template such that the barcode can identify the edit made to the corresponding cellular target sequence. The barcode typically comprises four or more nucleotides.

In certain embodiments, plasmid backbones and other editing vectors may further comprise one or more selectable markers to enable artificial selection of cells undergoing editing and/or curing events. For example, in certain embodiments, the plasmid backbones encode for one or more antibiotic resistance genes, such as ampicillin/carbenicillin and chloramphenicol resistance genes, thereby facilitating enrichment for cells undergoing editing and/or curing events via depletion of the cell population. In other examples, plasmid backbones may include an integrated GFP gene to enable phenotypic detection of editing and/or curing events by flow cytometry, fluorescent cell imaging, etc.

In certain embodiments, plasmid backbones and other editing vectors editing vectors may further comprise control sequences operably linked to the editing component sequences to be transcribed. As described above, promoters driving transcription of one or more components of the nucleic acid-guided nuclease editing system may be inducible. A number of gene regulation control systems have been developed for the controlled expression of genes in plant, microbe, and animal cells, including mammalian cells, such as the pL promoter (induced by heat inactivation of the cI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. In certain embodiments of the present methods used in the modules and instruments described herein, at least one of the nucleic acid-guided nuclease editing components (e.g., the nuclease and/or the gRNA) is under the control of a promoter that is activated by a rise in temperature, as such a promoter allows for the promoter to be activated by an increase in temperature, and de-activated by a decrease in temperature, thereby “turning off” the editing process. Thus, in the scenario of a promoter that is de-activated by a decrease in temperature, editing in the cell can be turned off without having to change media; to remove, e.g., an inducible biochemical in the medium that is used to induce editing.

Certain embodiments described herein also utilize an alternative to traditional nucleic acid-guided nuclease editing (i.e., RNA-guided nuclease or CRISPR editing) for performing genome editing. More particularly, such embodiments employ a nucleic acid-guided nickase/reverse transcriptase fusion enzyme (“nickase-RT fusion”) as opposed to a nucleic acid-guided nuclease (i.e., a “CRISPR nuclease”). Editing with a nickase-RT fusion differs from traditional CRISPR editing in that instead of initiating double-stranded breaks in the target genome and homologous recombination to effect an edit, the nickase initiates a nick in a single strand of the target genome, e.g., the non-complementary strand. Further, the fusion of the nickase to a reverse transcriptase, in combination with an editing cassette comprising a CFgRNA and repair template, e.g. a CF editing cassette, eliminates the need for a donor DNA to be incorporated by homologous recombination. Instead, the repair template of the corresponding cassette-typically a ribonucleic acid—may serve as a template for the reverse transcription (“RT”) portion of the fusion enzyme to add an intended to the nicked strand at the target locus. That is, utilization of a nickase-RT fusion enables incorporation of the edit in the target genome by copying an RNA sequence (i.e., at the RNA level) rather than replacing a portion of the target locus with a donor DNA (i.e., at the DNA level).

The nickase-functioning as a single-strand cutter and having the specificity of a nucleic acid-guided nuclease-engages the target locus and nicks a strand of the target locus creating one or more free 3′ terminal nucleotides. The 3′ end of the editing cassette is then annealed to the nicked strand, and the reverse transcriptase utilizes the 3′ terminal nucleotide(s) of the nicked strand to copy the repair template and create a “flap” containing the desired edit. Thereafter, endogenous repair mechanisms of the cells repair the nick in favor of the desired edit by hybridizing the flap to the wild-type (e.g., unedited) DNA strand.

Improved Nucleic Acid-Guided Nuclease Editing With DNA Repair Mechanism Disruption

The present disclosure is drawn to increasing the efficiency of nucleic acid-guided nuclease editing in cells via disruption of cellular DNA repair mechanisms, and in particular embodiments, DNA mismatch repair (MMR) mechanisms.

Recently, it has been reported that expression of MMR-related genes can inversely correlate with precise editing outcomes. See Chen et al., Cell 184, 1-18 (2021); da Silva et al., bioRxiv 2021.09.30.462548 (2021); See also, Wout et al., bioRxiv 2022.05.04.490422 (2022). More particularly, it has been reported that certain cellular MMR pathways may inhibit, or at least strongly suppress, precise nucleic acid-guided nuclease editing in live cells and may instead promote unwanted indel formation. See Chen et al., Cell 184, 1-18 (2021).

To mitigate the negative effects of MMR on editing outcomes, recent work has focused on MMR inhibition, or downregulation, via MMR-targeting siRNAs and other MMR-inhibiting agents. For example, Chen has reported that siRNA-mediated knockdown of certain MMR genes, as well as the expression of dominant negative MMR protein variants, may lead to greater editing efficiency (see Chen et al., Cell 184, 1-18 (2021). Similarly, da Silva has demonstrated that editing rates may be improved via the utilization of siRNAs to deplete MMR transcripts, and/or the utilization of protein degradation technologies, such as dTAG, to deplete MMR proteins (see da Silva et al., bioRxiv 2021.09.30.462548 (2021)). Da Silva has further demonstrated that cells lines comprising MMR gene knockouts may yield significantly higher editing efficiencies as compared to other cell lines. Da Silva et al., bioRxiv 2021.09.30.462548 (2021).

The inventors of the present disclosure, however, have discovered that overexpression of certain MMR proteins (or polypeptides), and especially overexpression of certain wild-type MMR proteins, also perturbs MMR mechanisms, such that when these MMR proteins are overexpressed during editing, the negative effects of associated MMR pathways on editing outcomes may be mitigated. This strategy, which seemingly contradicts the MMR inhibition and downregulation methods proposed by other contemporary studies (see Chen et al., Cell 184, 1-18 (2021); da Silva et al., bioRxiv 2021.09.30.462548 (2021); and, Wout et al., bioRxiv 2022.05.04.490422 (2022)), results in similar or even better editing efficiencies as compared to such MMR inhibition/downregulation methods.

Accordingly, embodiments of the present disclosure provide compositions of matter, methods, systems, and instruments for improved nucleic acid-guided nuclease editing, wherein cellular DNA repair perturbation agents comprising exogenous DNA repair polypeptides and/or proteins, or polynucleotides encoding the DNA repair polypeptides and/or proteins, are co-delivered into and/or are co-expressed in cells along with editing machinery, e.g., editing gRNAs and corresponding nucleases or nickases. The expression/overexpression of such DNA repair polypeptides and/or proteins in the cells perturbs/disrupts native cellular DNA repair mechanisms, such as native MMR pathways, that can negatively affect editing outcomes, thereby resulting in more precise editing and a reduction in reversion to wild-type sequence or unwanted indel formation.

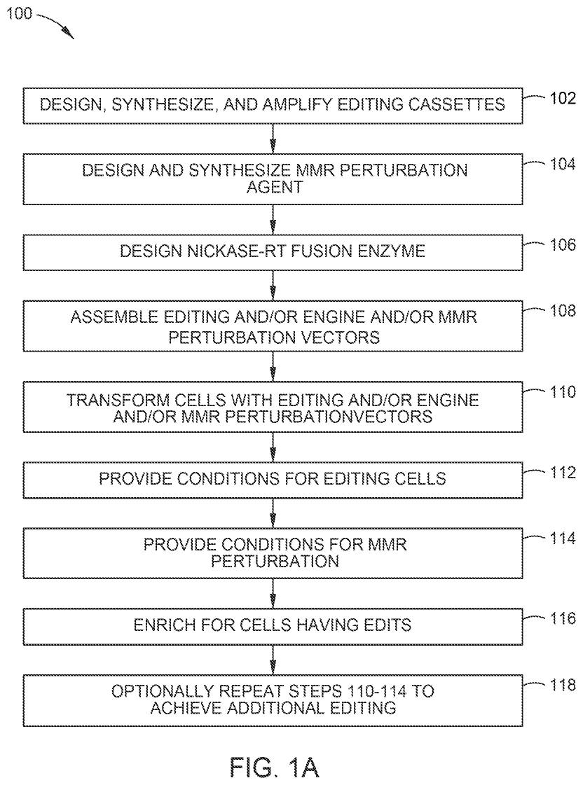

FIG. 1A is a simplified block diagram of an exemplary method 100 for improved editing of live cells via nucleic acid-guided nuclease editing, such as nickase/reverse transcriptase fusion (“nickase-RT fusion”) editing, with simultaneous disruption of cellular DNA repair mechanisms. More particularly, the exemplary method 100 utilizes a nickase-RT fusion enzyme and CREATE fusion editing cassette (“CF editing cassette”), in combination with an MMR perturbation agent, to effect an intended edit in the cell genome while disrupting one or more associated MMR pathways of the cells. Though described with reference to CREATE fusion Editing (CFE), the exemplary method 100 may also apply to other types of nucleic acid-guided nuclease editing compositions, components, and systems. Similarly, though MMR disruption is described in method 100, other DNA repair pathways and/or repair proteins are also contemplated, including base excision repair (BER), nucleotide excision repair (NER), homologous recombination (HR), non-homologous end joining (NHEJ), as well as other DNA repair pathways and/or mechanisms.

Looking at FIG. 1A, method 100 begins at 102 by designing and synthesizing one or more CF editing cassettes, such as a library of CF editing cassettes, each cassette comprising a CFgRNA having a covalently-linked editing gRNA and repair template designed to incorporate at least one edit into one or both DNA strands at a first target locus. That is, each CFgRNA of a CF editing cassette comprises an editing gRNA sequence and a repair template sequence to be reverse transcribed, wherein the repair template sequence comprises a desired genome edit. In certain embodiments, the CF editing cassettes may further include a PAM and/or spacer mutation(s). Once the CF editing cassettes are synthesized, individual cassettes may be amplified.

At 104, an MMR perturbation agent is designed and synthesized. Generally, the MMR perturbation agent acts to disrupt, or even inhibit, one or more MMR pathways of the cells being edited. In certain embodiments, the MMR perturbation agent comprises one or more polypeptides and/or proteins that facilitate disruption or inhibition of the one or more MMR pathways of the cells, or polynucleotides encoding such polypeptides and/or proteins which may be amplified after synthesis. The polypeptide(s) and/or protein(s) may comprise naturally occurring (e.g., wild-type) polypeptides and/or proteins of the cells, and/or non-naturally occurring (e.g., a variant) polypeptides and/or proteins. In certain embodiments, the polypeptide(s) and/or protein(s) comprise dominant-negative variants, or other non-functional or catalytically-inactive mutants. In certain embodiments, the MMR perturbation agent comprises a combination of naturally occurring and non-naturally occurring polypeptides and/or proteins.

In certain embodiments, the MMR perturbation agent comprises polypeptides and/or proteins that are indirectly or directly involved in an MMR pathway of the cells. For example, the MMR perturbation agent may comprise one or more MMR proteins, or one or more portions of MMR proteins, such as one or more domains of MMR proteins. Examples of suitable MMR proteins for use with the present disclosure include MLH1, MLH1co, MLH2, MLH3, MSH2, MSH3, MSH6, PMS1, PMS2, variants thereof, portions thereof, homologs thereof, orthologs thereof, and the like. However, still other MMR proteins are also contemplated.

In further embodiments, the MMR perturbation agent comprises an MMR complex (e.g., dimer, trimer, etc.) having two or more MMR proteins or portions of MMR proteins, or a portion of an MMR complex. For example, the MMR perturbation agent may comprise a MutL and/or MutS complex, e.g., MutLα, MutLβ, MutLγ, MutSα, MutSβ, etc. However, still other complexes are also contemplated. Note that although many of the presently-described examples include human proteins, such repair proteins are generally conserved or at least present in some form across a wide variety of species, including microbes such as E. coli. Accordingly, the described examples may be utilized for editing a variety of different cell types in addition to human/mammalian cells, including bacterial and fungal cells.

In certain embodiments, in addition or alternative to MMR proteins, the MMR perturbation agent comprises one or more other polypeptides, proteins, and/or portions thereof involved in DNA replication, recombination, or repair processes, or polynucleotides encoding such polypeptides, proteins, and/or portions that may facilitate disruption or inhibition of cellular MMR pathways. Examples of such proteins include MEAF6, MPG, LIG1, LIG3 (A9), LIG3 (B5), FEN1, MRGBP, KAT7, RPA2, CHAF1B, ALKBHZ, NUDT1 (B4), BUDT1 (B8), variants thereof, portions thereof, homologs thereof, orthologs thereof, and the like.

In embodiments where the MMR perturbation agent comprises exogenous polynucleotides encoding polypeptides and/or proteins, such polynucleotides may include an open reading frame (ORF) of an MMR protein. In certain embodiments, the exogenous polynucleotides comprise in vitro transcribed MMR protein mRNA.

In certain embodiments, the exogenous polynucleotides may further include promoters at 5′ ends of the encoded polypeptides or proteins for transcriptional control thereof. In certain embodiments, the promoters comprise inducible promoters, such as chemically-inducible promoters, temperature-inducible promoters, light-inducible promoters, and the like. In certain embodiments, the promoters comprise constitutive promoters. Generally, the promoters may include the same or different promoters as utilized with other editing components of the present method, such as the CF editing cassettes and/or CF enzymes. In further embodiments, the polynucleotides further include nuclear localization sequences (NLS), which may be at the 5′ or 3′ ends of the encoded polypeptides and/or proteins.

At 106, a nickase-RT fusion enzyme (e.g., a CF enzyme) is designed. As described above, the nickase-RT fusion enzyme comprises, in order from amino terminus to carboxy terminus, or from carboxy terminus to amino terminus, a nucleic acid-guided nickase and a reverse transcriptase. The nickase-RT fusion enzyme may be delivered to the cells as a coding sequence in a vector (in some embodiments under the control of an inducible promoter), such as the same or different vector as the CF editing cassette(s) and/or polynucleotide(s) of the MMR perturbation agent, or the nickase-RT fusion enzyme may be delivered to the cells as a protein or protein complex.

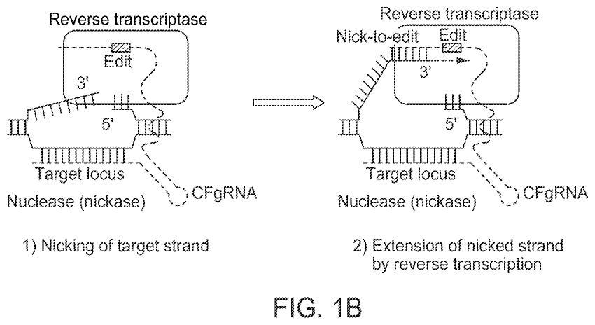

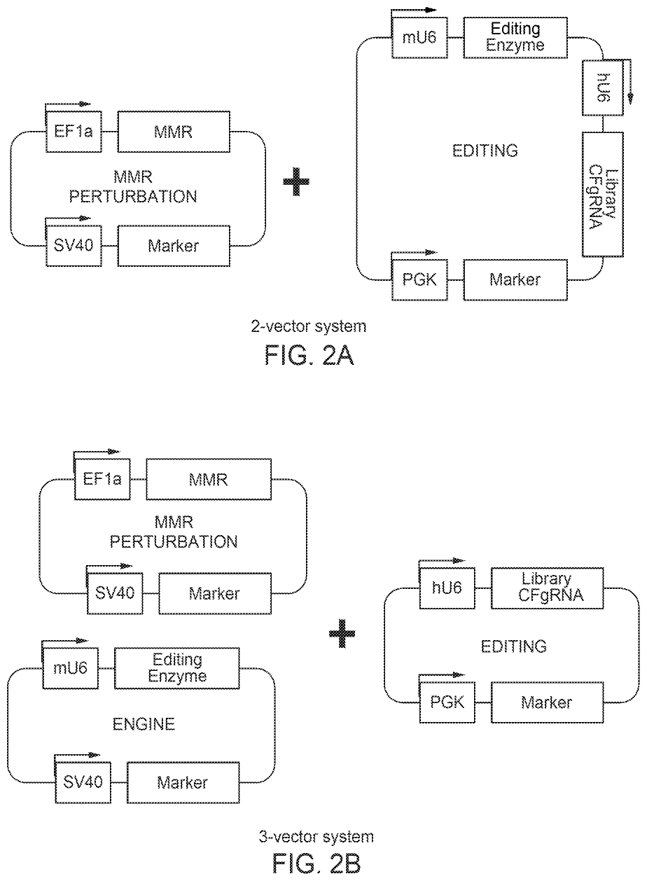

At 108, the CF editing cassette(s), the nickase-RT fusion enzyme, and in certain embodiments, the MMR perturbation agent (e.g., as one or more polynucleotides), are assembled with vector backbones, such as plasmid backbones, to create “editing” vectors, and/or “engine” vectors, and/or “MMR perturbation vectors,” e.g., a library of vectors. In certain embodiments, a CF editing cassette, nickase-RT fusion enzyme, and polynucleotide(s) encoding MMR-perturbing polypeptides and/or proteins are assembled together on a single vector. In certain embodiments, the CF editing cassette and nickase-RT fusion enzyme are assembled into an editing vector together, and the polynucleotide(s) encoding the MMR-perturbing polypeptides and/or proteins are assembled into separate, MMR perturbation vectors. An example of such a two-vector system is illustrated in FIG. 2A. In other embodiments, however, the polynucleotide(s) encoding the MMR-perturbing polypeptides and/or proteins and the nickase-RT fusion enzyme are assembled into a vector together, and the CF editing cassette is assembled into a separate editing vector. In still other embodiments, the CF editing cassette, the polynucleotide(s) encoding the MMR-perturbing polypeptides and/or proteins, and the nickase-RT fusion enzyme are each assembled into separate editing, MMR perturbation, and engine vectors, respectively. An example of such a three-vector system is illustrated in FIG. 2A. In embodiments where the perturbation agent comprises polypeptides and/or proteins, only the CF editing cassette and/or the nickase-RT fusion enzyme may be assembled with vector backbones at 108.

At 110, the editing vectors, engine vectors, and/or MMR perturbation vectors (or MMR-pertubing polypeptides or proteins) are introduced into the live cells. A variety of delivery systems may be used to introduce (e.g., transform, transfect, or transduce) nucleic acid-guided nickase fusion editing system components and MMR perturbation agent into a host cell 110. These delivery systems include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of particular interest is the use of electroporation, particularly flow-through electroporation (either as a stand-alone instrument or as a module in an automated multi-module system) as described in, e.g., U.S. Pat. Nos. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; U.S. Pat. No. 10,738,663, issued 29 Sep. 2020; and U.S. Pat. No. 10,894,958, issued 19 Jan. 2021 all of which are herein incorporated by reference in their entirety.

Once transformed 110, the next steps in method 100 include providing conditions for nucleic acid-guided nuclease editing 112 and for MMR perturbation 114. As described above, the inventors of the present disclosure have discovered that overexpression of certain MMR (or other) proteins or portions thereof disrupts and in certain cases, inhibits, associated cellular MMR pathways. Accordingly, when these MMR proteins are expressed/overexpressed before or during editing, negative effects of associated MMR mechanisms on editing may be suppressed or avoided, resulting in improved editing outcomes.

Generally, “providing conditions” at 110 includes incubation of the cells in appropriate medium and may also include providing conditions to induce transcription of an inducible promoter (e.g., adding antibiotics, adding inducers, increasing temperature) for transcription of a CF editing cassette, nickase-RT fusion enzyme, and in certain embodiments, polynucleotide(s) encoding the MMR-perturbing polypeptides and/or proteins. In certain embodiments, the conditions for editing 112 and for perturbation 114 (e.g., MMR-perturbing protein expression/overexpression) are the same and thus, these steps are performed simultaneously. In certain embodiments, the conditions for editing 112 and for MMR perturbation 114 are different (e.g., an MMR-perturbing protein sequence may be under the control of a different inducible promoter than the components of the editing system), and these steps may be performed either simultaneously or in sequence.

Once editing is complete, the cells are allowed to recover and are preferably enriched for cells that have been edited 116. Enrichment can be performed directly, such as via cells from the population that express a selectable marker, or by using surrogates, e.g., cell surface handles co-introduced with one or more components of the editing components. At this point in method 100, the cells can be characterized phenotypically or genotypically, e.g., via sequencing, or, optionally, any of steps 102-116 or steps 110-116 may be repeated to make additional edits 118 in recursive or iterative editing rounds. In certain embodiments, steps 102-116 are repeated to create or construct a defined combination of edits or a combinatorial library.

For reference, FIG. 1B is a simplified graphic depiction of the mechanism of a nucleic acid-guided nickase enzyme/reverse transcriptase fusion enzyme edit. At left in FIG. 1B, a nickase-RT fusion enzyme and CFgRNA of a CF editing cassette are shown bound to a target locus of the cell genome, where the target locus in the context of the methods and compositions herein is a locus of approximately 1 to 1,000 nucleotides or more in length, or 2 to 500 nucleotides in length, or 10 to 400 nucleotides in length, or 20 to 200 nucleotides in length. In one step, the nickase-RT fusion enzyme and the CFgRNA bind to the target locus and the nickase nicks a single DNA strand at the target locus, thus creating a 3′ “flap.” In order for the nickase-RT fusion enzyme and the CFgRNA to bind to the target locus and nick the genomic DNA, there must be a protospacer adjacent motif (PAM) appropriately located in or adjacent (e.g., downstream) to the target locus and on the strand to be nicked and edited. The CFgRNA must also be complementary to a region of the strand to be edited and must include the desired edit to be incorporated.

At right in FIG. 1B shows the previously formed flap, where the reverse transcriptase (RT) portion of the nickase-RT fusion enzyme adds nucleotides to extend the 3′ free end of the nicked strand using the repair template of the CFgRNA, which includes the desired edit, as a template. The regions of the DNA strands that are synthesized by the RT may include, e.g., a nick-to-edit region, an edit region, and a post-edit homology (PEH) region. The nick-to-edit region and the post-edit homology (PEH) region are complementary to the unedited (i.e., wild-type) strand, thus facilitating resolution of the edited flap with the unedited strand via endogenous repair mechanisms, e.g., homology-directed repair (HDR), recombination pathways, or other DNA repair pathways. The target locus may resolve into either wild-type, where the desired edit is not incorporated, or into an edited target locus. Once the DNA flap containing the edit is synthesized, an equilibrium is established between the newly synthesized 3′ flap and the wild-type 5′ flap. The equilibrium can be affected by the length of the edit, nick-to-edit distance, and/or post edit homology region. In order for the newly synthesized flap to be incorporated into the genome, the 5′ flap is likely degraded by an exonuclease. This allows the 3′ flap to anneal to the DNA, and a polymerase then likely fills in any missing nucleotides and a DNA ligase seals the nick.

At this stage, one DNA strand contains the edit while the second DNA strand does not. A DNA replication process or similar process is likely responsible for copying the edit into both strands. Note that DNA replication and associated processes, including MMR and other cellular DNA repair processes, may favor the wild-type strand as opposed to the edited strand after annealing. For example, MMR machinery may recognize the mismatch between the edited strand and the wild-type strand, excise the “error-prone” edited strand, and thereafter fill in/seal the gap via templated repair using the wild-type strand, thus reverting the edited strand to wild-type, and decreasing editing efficiency of the editing process. Accordingly, editing and repair or reversion to wild-type are constantly in competition, wherein the outcome may be determined by cellular detection and repair response. To overcome or curtail these cellular repair and reversion mechanisms, a DNA repair perturbation agent, such as an MMR perturbation agent, may be introduced into the cells prior to or during editing, which can disrupt such cellular mechanisms and improve editing outcomes, as described above. By disrupting or inhibiting MMR and other cellular DNA repair mechanisms, editing, such as CF editing, may be favored as compared to repair or reversion of edited strands to wild-type.

FIG. 2A schematically depicts an exemplary two-vector system for nickase-RT fusion editing (CF editing) of live cells according to embodiments described herein. Note that the layouts of the vectors in FIG. 2A are only exemplary and do not limit embodiments of the present disclosure to any particular arrangement or orientation of components. For example, in certain other embodiments, the CF editing components and MMR perturbation agent may be disposed on a single vector.

As shown, the “editing” vector comprises a nickase-RT fusion enzyme and a CF editing cassette comprising a CFgRNA. Meanwhile, the “MMR perturbation” vector comprises a polynucleotide encoding an MMR-perturbing polypeptide or protein (“MMR”), which may be a wild-type MMR protein of the cells. In certain embodiments, one or both vectors of the two-vector system further include a selectable marker to facilitate enrichment or selection of cells successfully transformed with each respective vector, e.g., while performing the method 100. Accordingly, the selectable marker(s) may be used to “tag” and enrich for transformation events. Examples of suitable markers include antibiotic resistance genes and fluorescent proteins, such as the PuroR gene.

Also shown in both vectors are one or more promoters (e.g., inducible or constitutive), which may be integrated into the vector at 5′ ends of the nickase-RT fusion enzyme, the CF editing cassette, the MMR-perturbing protein, the selectable marker(s), and/or other components to drive transcription thereof. For example, in the editing vector of FIG. 2A, the nickase-RT fusion enzyme is depicted as being under the transcriptional control of a mouse U6 (“mU6”) promoter, the CF editing cassette is under the transcriptional control of a human U6 (“hU6”) promoter, and the selectable marker is under the transcriptional control of a phosphoglycerate kinase (“PGK”) promoter. Similarly, in the MMR perturbation vector, the MMR protein is shown as being under the control of a eukaryotic translation elongation factor 1 α (“EF1A”) promoter, and the selectable marker is under the control of a simian virus 40 (“SV40”) promoter. However, other promoters are also contemplated.

FIG. 2B schematically depicts an exemplary three-vector system for nickase-RT fusion editing of live cells according to embodiments described herein. In particular, FIG. 2B depicts a system wherein each of a nickase-RT fusion enzyme, a CF editing cassette comprising a CFgRNA, and a polynucleotide encoding an MMR-perturbing polypeptide or protein are assembled into separate vectors. Again, the layout of the three vectors in FIG. 2B is only exemplary, and does not limit embodiments of the present disclosure to any particular arrangement or orientation of components.

Similar to previous example, the nickase-RT fusion enzyme, the CF editing cassette, the MMR-perturbing protein, and/or other components of the multiple vectors may be under the control of one or more inducible or constitutive promoters. For purposes of illustration, the same promoters in FIG. 2A are depicted in FIG. 2B. However, other promoters are also contemplated.

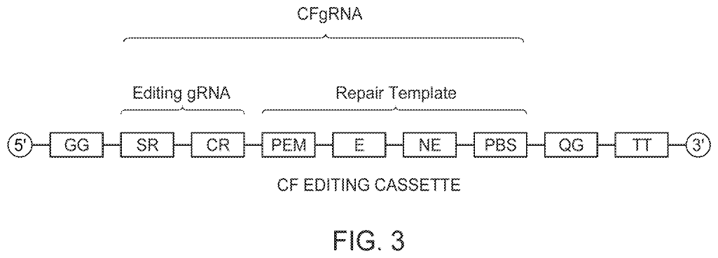

FIG. 3 is a simplified graphic depiction of an exemplary double-stranded CF editing cassette for nickase-RT fusion editing of live cells. Note that the layout of the cassette in FIG. 3 is only exemplary and does not limit embodiments of the present disclosure to any particular arrangement or orientation of components thereof. In FIG. 3, the CF editing cassette comprises a CFgRNA having an editing gRNA covalently linked to a repair template. More particularly, the CF editing cassette comprises from 5′ to 3′ an optional transcription initiation sequence which facilitates high efficiency transcription (e.g., by T7 RNA polymerase) (denoted “GG”); a CFgRNA comprising from 5′ to 3′ an editing gRNA configured to target a first target locus and comprising a spacer region (denoted “SR”) having complementarity to the first target locus and a scaffold or repeat region (denoted “CR”) for complexing with a nickase-RT fusion enzyme, as well as a repair template comprising from 5′ to 3′ an optional post-edit homology region (denoted “PEH”), an edit (denoted “E”), a nick-to-edit region (denoted “NE”), and a primer binding site (denoted “PBS”); an optional G-quadruplex region (denoted “QG”) or other nucleic acid stabilization moiety for stabilizing the CF editing cassette preventing degradation of the repair template; and a PolyT transcription terminator (denoted “TT”).

### Examples

The following examples are put forth so as to provide those of ordinary skill in the art with a complete disclosure and description of how to make and use the present disclosure, and are not intended to limit the scope of what the inventors regard as their disclosure, nor are they intended to represent or imply that the experiments below are all of or the only experiments performed. It will be appreciated by persons skilled in the art that numerous variations and/or modifications may be made to the disclosure as shown in the specific aspects without departing from the spirit or scope of the disclosure as broadly described. The present aspects are, therefore, to be considered in all respects as illustrative and not restrictive.

### Example I: GFP to BFP Conversion Assay

A GFP to BFP reporter cell line was created using mammalian cells with a stably integrated genomic copy of the GFP gene (HEK293T-GFP). These cell lines enabled phenotypic detection of genomic edits of different classes by various different mechanisms, including flow cytometry, fluorescent cell imaging, and genotypic detection by sequencing of the genome-integrated GFP gene. Lack of editing, or perfect repair of cut events in the GFP gene, result in cells that remain GFP-positive. Cut events that are repaired by the Non-Homologous End-Joining (NHEJ) pathway often result in nucleotide insertion or deletion events (indels), resulting in frame-shift mutations in the coding sequence that cause loss of GFP gene expression and fluorescence. Cut events that are repaired by the Homology-Directed Repair (HDR) pathway, using the GFP-to-BFP HDR donor as a repair template or by the use of CFgRNAs, result in conversion of the cell fluorescence profile from that of GFP to that of BFP.

### Example II: CREATE Fusion Editing

CREATE fusion editing (CFE) is a live cell editing technique that uses a nucleic acid-guided nickase fusion protein (e.g., MAD2007 nickase and others, see U.S. Pat. Nos. 10,883,077; 11,053,485; and 11,085,030; and U.S. Ser. Nos. 17/200,089 and 17/200,110 filed 12 Mar. 2021; Ser. No. 17/463,498, filed 23 Aug. 2021; and Ser. No. 17/463,581, filed 1 Sep. 2021) fused to a peptide with reverse transcriptase activity along with a nucleic acid encoding a CFgRNA (i.e., CF editing cassette) comprising a region complementary to a target region of a nucleic acid in one or more cells, which comprises a mutation of at least one nucleotide relative to the target region in the one or more cells and a protospacer adjacent motif (PAM) mutation.

In a first design, a nickase enzyme derived from the MAD2007 nuclease (see, U.S. Pat. Nos. 9,982,279 and 10,337,028), e.g., Cas9 H840A nickase or MAD7 nickase (see, e.g., U.S. Ser. Nos. 16/837,212 and 17/084,522), was fused to an engineered reverse transcriptase (RT) on the C-terminus and cloned downstream of a CMV promoter. In this instance, the RT used was derived from Moloney Murine Leukemia Virus (M-MLV).

CF editing cassettes were designed comprising CFgRNAs that were complementary to a single region proximal to the EGFP-to-BFP editing site. The repair template on the 3′ end included a region of 13 bp that include the TY-to-SH edit and a second region of 13 bp that is complementary to the nicked EGFP DNA sequence. This allowed the nicked genomic DNA to anneal to the 3′ end of the repair template which can then be extended by the reverse transcriptase to incorporate the edit in the genome. A second CF editing cassette targeted a region in the EGFP DNA sequence that is 86 bp upstream of the edit site. The CFgRNA of the second CF editing cassette was designed such that it enables the nickase to cut the opposite strand relative to the other CF editing cassette. Both of these CF editing cassettes were cloned downstream of a U6 promoter. A poly-T sequence was also included that terminates the transcription of the CF editing cassette.

The plasmids were transformed into NEB Stable E. coli (Ipswich, NY) and grown overnight in 25 mL LB cultures. The following day the plasmids were purified from E. coli using the Qiagen Midi Prep kit (Venlo, Netherlands). The purified plasmid was then RNase A (ThermoFisher, Waltham, Mass) treated and re-purified using the DNA Clean and Concentrator kit (Zymo, Irvine, CA).

HEK293T cells were cultured in DMEM medium which was supplemented with 10% FBS and 1× Penicillin and Streptomycin. 100 ng of total DNA (50 ng of gRNA plasmid and 50 ng of CFE plasmids) was mixed with 1 μl of PolyFect (Qiagen, Venlo, Netherlands) in 25 μl of OptiMEM in a 96 well plate. The complex was incubated for 10 minutes and then 20,000 HEK293T cells resuspended in 100 μl of DMEM were added to the mixture. The resulting mixture was then incubated for 80 hours at 37 C and 5% CO₂.

The cells were harvested from flat bottom 96 well plates using TrypLE Express reagent (ThermoFisher, Waltham, Mass) and transferred to v-bottom 96 well plate. The plate was then spun down at 500 g for 5 minutes. The TrypLE solution was then aspirated and the cell pellet was resuspended in FACS buffer (1×PBS, 1% FBS, 1 mM EDTA and 0.5% BSA). The GFP+, BFP+ and RFP+ cells were then analyzed on the Attune N×T flow cytometer and the data was analyzed on FlowJo software.

The RFP+BFP+ cells that were identified were indicative of the proportion of enriched cells that have undergone precise or imprecise editing process. BFP+ cells indicate cells that have undergone successful editing process and express BFP. The GFP-cells indicate cells that have been imprecisely edited, leading to disruption of the GFP open reading frame and loss of expression.

In this exemplary experiment, the edit is positioned roughly 5′ in the repair template and 3′ of the edit is a further region complementary to the nicked genome, although the intended edit could also be present further within the region homologous to the nicked genome. A nickase RT fusion enzyme (Cas9 H840A nickase or MAD2007) created a nick in the target site and the nicked DNA annealed to its complementary sequence on the 3′ end of the repair template. The RT then extended the DNA, thereby incorporating the intended edit directly in the genome.

The effectiveness of CREATE fusion editing in GFP+ HEK293T cells was then tested. In the assay system devised, a successful precise edit resulted in a BFP+ cell whereas an imprecisely edited cells turned the cell both BFP and GFP negative. CREATE Fusion gRNA in combination with CFE2.1 or CFE2.2 gave ˜40-45% BFP+ cells indicating that almost half the cell population has undergone successful editing (data not shown). The GFP− cells are ˜10% of the population. The use of a second nicking gRNA, as described in Liu et al. (Nature, 576(7785):149-157 (2019)) did not increase the precision edit rate any further; in fact, it significantly increased the imprecisely edited, GFP-negative cell population and the editing rate was lower.

Previous literature has shown that double nicks on opposite strands (<90 bp away) do result in a double strand break which tend to be repaired via NHEJ resulting in imprecise insertions or deletions. Overall, the results indicated that CREATE fusion editing predominantly yielded precisely edited cells and the imprecisely edited cells proportion is much lower (data not shown).

An enrichment handle, specifically a fluorescent reporter (RFP) linked to nuclease expression was included in this experimentation as a proxy for cells receiving the editing machinery. When only the RFP-positive cells were analyzed (computational enrichment) after 3-4 cell divisions, up to 75% of the cells were BFP+ when tested with CF editing cassettes (data not shown), indicating uptake or expression-linked reporters can be used to enrich for a population of cells with higher rates of CREATE fusion-mediated gene editing. In fact, the combined use of CREATE fusion editing and the described enrichment methods resulted in a significantly improved rate of intended edits (data not shown).

### Example III: CREATE Fusion Editing with CF Editing Cassette

CREATE fusion editing was carried out in mammalian cells using a CF editing cassette comprising a CFgRNA having an intended edit to the native sequence and an edit that disrupts nuclease cleavage at this site. Briefly, lentiviral vectors were produced using the following protocol: 1000 ng of Lentiviral transfer plasmid containing the editing cassettes along with 1500 ng of Lentiviral Packaging plasmids (ViraSafe Lentivirus Packaging System Cell BioLabs) were transfected into HEK293T cells using Lipofectamine LTX in 6-well plates. Media containing the lentivirus was collected 72 hrs post transfection. Two clones of a lentiviral CF editing cassette design were chosen, and an empty lentiviral backbone was included as negative control.

The day before the transduction, 200,000 HEK293T cells were seeded in six well plates. Different volumes of CF editing cassette lentivirus (10 to 1000 μl) were added to HEK293T cells in 6-well plates along with 10 μg/ml of Polybrene. 48 hours after transduction, media with 15 μg/ml of Blasticidin was added to the wells. Cells were maintained in selection for one week. Following selection, the well with lowest number of surviving cells was selected for future experiments (<5% cells).

The experimental constructs or wild-type SpCas9 were electroporated into HEK293T cells using the Neon Transfection System (Thermo Fisher Scientific, Waltham, MA, USA). Briefly, 400 ng of total plasmid DNA was mixed with 100,000 cells in Buffer R in a total of 15μl volume. The 10 μl Neon tip was used to electroporate cells using 2 pulses of 20 ms and 1150 v. Cells were analyzed on the flow cytometer 80 hrs post electroporation. Unenriched editing rates of up to 15% were achieved from single copy delivery of an editing cassette (data not shown).

When the editing was combined with computational selection of RFP+ cells, however, enriched editing rates of up to 30% were achieved from a single copy delivery CF editing cassette. This enrichment via selection of cells receiving the editing machinery was shown to result in a 2-fold increase in precise, complete intended edits (data not shown). Two or more enrichment/delivery steps can also be used to achieve higher editing rates of CREATE fusion editing in an automated instrument, e.g., use of a module for cell handle enrichment and identification of cells having BFP expression. When the method enriched for cells that have higher CF editing cassette expression levels, the editing rate was even further increased, and thus a growth and/or enrichment module of the instrument may include editing cassette enrichment.

### Example IV: Screening MMR Proteins for Enhanced CREATE Fusion Editing

CREATE fusion editing was carried out in iPSCs to identify MMR proteins that, when expressed during CF editing, would improve GFP-to-BFP edit rates. Here, a two-vector editing system was used, wherein the two-vector system included a first “editing” plasmid comprising a nucleotide sequence encoding one of four CFgRNAs (“g1,” “g18,” “g25,” or “g5”) and a corresponding CF enzyme to incorporate a GFP-to-BFP edit, and a second “MMR expression” plasmid comprising a nucleotide sequence encoding one of forty-eight (48) MMR constructs comprising MMR proteins, as listed in Table 1 found below, to perturb an associated MMR pathway.

TABLE 1

| | | | | | Avg Fold- | |
|---|---|---|---|---|---|---|
| Construct | Construct | | Base | Variant | Improvement | AA SEQ |
| No. | description | Base variant | Gene | Type | Over Control | ID |
| pMMR01 | pR-wtMSH2- | wtMSH2 | MSH2 | wild-type | 2.37 | 1 |
| | mCherry | | | ORF | | |
| pMMR02 | pR-NLS(cMyc)- | wtMSH2 | MSH2 | wild-type | 2.8 | 2 |
| | wtMSH2- | | | ORF | | |
| | mCherry | | | | | |
| pMMR03 | pR-wtMSH2- | wtMSH2 | MSH2 | wild-type | 2.42 | 3 |
| | NLS(SV40)- | | | ORF | | |
| | mCherry | | | | | |
| pMMR04 | pR-wtMLH1- | wtMLH1 | MLH1 | wild-type | 3.34 | 4 |
| | mCherry | | | ORF | | |
| pMMR05 | pR-NLS(cMyc)- | wtMLH1 | MLH1 | wild-type | 3.09 | 5 |
| | wtMLH1- | | | ORF | | |
| | mCherry | | | | | |
| pMMR06 | pR-wtMLH1- | wtMLH1 | MLH1 | wild-type | 3.1 | 6 |
| | NLS(SV40)- | | | ORF | | |
| | mCherry | | | | | |
| pMMR07 | pR-wtMSH6- | wtMSH6 | MSH6 | wild-type | 1.32 | 7 |
| | mCherry | | | ORF | | |
| pMMR08 | pR-NLS(cMyc)- | wtMSH6 | MSH6 | wild-type | 1.27 | 8 |
| | wtMSH6- | | | ORF | | |
| | mCherry | | | | | |
| pMMR09 | pR-wtMSH6- | wtMSH6 | MSH6 | wild-type | 1.23 | 9 |
| | NLS(SV40)- | | | ORF | | |
| | mCherry | | | | | |
| pMMR10 | pR-wtPMS2- | wtPMS2 | PMS2 | wild-type | 1.29 | 10 |
| | mCherry | | | ORF | | |
| pMMR11 | pR-NLS(cMyc)- | wtPMS2 | PMS2 | wild-type | 1.2 | 11 |
| | wtPMS2- | | | ORF | | |
| | mCherry | | | | | |
| pMMR12 | pR-wtPMS2- | wtPMS2 | PMS2 | wild-type | 1.05 | 12 |
| | NLS(SV40)- | | | ORF | | |
| | mCherry | | | | | |
| pMMR13 | pR- | MSH2_K675R | MSH2 | point | 2.97 | 13 |
| | MSH2_K675R- | | | mutant | | |
| | mCherry | | | | | |
| pMMR14 | pR-NLS(cMyc)- | MSH2_K675R | MSH2 | point | 3.68 | 14 |
| | MSH2_K675R- | | | mutant | | |
| | mCherry | | | | | |
| pMMR15 | pR- | MSH2_K675R | MSH2 | point | 2.97 | 15 |
| | MSH2_K675R- | | | mutant | | |
| | NLS(SV40)- | | | | | |
| | mCherry | | | | | |
| pMMR16 | pR- | MSH2_K675A | MSH2 | point | 2.62 | 16 |
| | MSH2_K675A- | | | mutant | | |
| | mCherry | | | | | |
| pMMR17 | pR-NLS(cMyc)- | MSH2_K675A | MSH2 | point | 2.17 | 17 |
| | MSH2_K675A- | | | mutant | | |
| | mCherry | | | | | |
| pMMR18 | pR- | MSH2_K675A | MSH2 | point | 2.23 | 18 |
| | MSH2_K675A- | | | mutant | | |
| | NLS(SV40)- | | | | | |
| | mCherry | | | | | |
| pMMR19 | pR- | MSH2_M688R | MSH2 | point | 1.08 | 19 |
| | MSH2_M688R- | | | mutant | | |
| | mCherry | | | | | |
| pMMR20 | pR-NLS(cMyc)- | MSH2_M688R | MSH2 | point | 1.23 | 20 |
| | MSH2_M688R- | | | mutant | | |
| | mCherry | | | | | |
| pMMR21 | pR- | MSH2_M688R | MSH2 | point | 1.36 | 21 |
| | MSH2_M688R- | | | mutant | | |
| | NLS(SV40)- | | | | | |
| | mCherry | | | | | |
| pMMR22 | pR- | MSH2_ΔCTD | MSH2 | domain | 2.06 | 22 |
| | MSH2_ΔCTD- | | | truncation | | |
| | mCherry | | | | | |
| pMMR23 | pR-NLS(cMyc)- | MSH2_ΔCTD | MSH2 | domain | 1.94 | 23 |
| | MSH2_ΔCTD- | | | truncation | | |
| | mCherry | | | | | |
| pMMR24 | pR- | MSH2_ΔCTD | MSH2 | domain | | 24 |
| | MSH2_ΔCTD- | | | truncation | | |
| | NLS(SV40)- | | | | | |
| | mCherry | | | | | |
| pMMR25 | pR- | MLH1_NTD | MLH1 | domain | 2.66 | 25 |
| | MLH1_NTD- | | | truncation | | |
| | mCherry | | | | | |
| pMMR26 | pR-NLS(cMyc)- | MLH1_NTD | MLH1 | domain | 2.95 | 26 |
| | MLH1_NTD- | | | truncation | | |
| | mCherry | | | | | |
| pMMR27 | pR- | MLH1_NTD | MLH1 | domain | | 27 |
| | MLH1_NTD- | | | truncation | | |
| | NLS(SV40)- | | | | | |
| | mCherry | | | | | |
| pMMR28 | pR- | MSH6_F432A | MSH6 | point | 0.98 | 28 |
| | MSH6_F432A- | | | mutant | | |
| | mCherry | | | | | |
| pMMR29 | pR-NLS(cMyc)- | MSH6_F432A | MSH6 | point | 1.85 | 29 |
| | MSH6_F432A- | | | mutant | | |
| | mCherry | | | | | |
| pMMR30 | pR- | MSH6_F432A | MSH6 | point | 1.96 | 30 |
| | MSH6_F432A- | | | mutant | | |
| | NLS(SV40)- | | | | | |
| | mCherry | | | | | |
| pMMR31 | pR- | MSH6_K1140R | MSH6 | point | 1.9 | 31 |
| | MSH6_K1140R- | | | mutant | | |
| | mCherry | | | | | |
| pMMR32 | pR-NLS(cMyc)- | MSH6_K1140R | MSH6 | point | 1.4 | 32 |
| | MSH6_K1140R- | | | mutant | | |
| | mCherry | | | | | |
| pMMR33 | pR- | MSH6_K1140R | MSH6 | point | 1.4 | 33 |
| | MSH6_K1140R- | | | mutant | | |
| | NLS(SV40)- | | | | | |
| | mCherry | | | | | |
| pMMR34 | pR- | MSH6_M1153R | MSH6 | point | 0.94 | 34 |
| | MSH6_M1153R- | | | mutant | | |
| | mCherry | | | | | |
| pMMR35 | pR-NLS(cMyc)- | MSH6_M1153R | MSH6 | point | 1.17 | 35 |
| | MSH6_M1153R- | | | mutant | | |
| | mCherry | | | | | |
| pMMR36 | pR- | MSH6_M1153R | MSH6 | point | 1.37 | 36 |
| | MSH6_M1153R- | | | mutant | | |
| | NLS(SV40)- | | | | | |
| | mCherry | | | | | |
| pMMR37 | pR- | MSH6_ΔCTD | MSH6 | domain | 1.57 | 37 |
| | MSH6_ΔCTD- | | | truncation | | |
| | mCherry | | | | | |
| pMMR38 | pR-NLS(cMyc)- | MSH6_ΔCTD | MSH6 | domain | 1.51 | 38 |
| | MSH6_ΔCTD- | | | truncation | | |
| | mCherry | | | | | |
| pMMR39 | pR- | MSH6_ΔCTD | MSH6 | domain | | 39 |
| | MSH6_ΔCTD- | | | truncation | | |
| | NLS(SV40)- | | | | | |
| | mCherry | | | | | |
| pMMR40 | pR- | PMS2_ΔNTD | PMS2 | domain | 1.91 | 40 |
| | PMS2_ΔNTD- | | | truncation | | |
| | mCherry | | | | | |
| pMMR41 | pR-NLS(cMyc)- | PMS2_ΔNTD | PMS2 | domain | | 41 |
| | PMS2_ΔNTD- | | | truncation | | |
| | mCherry | | | | | |
| pMMR42 | pR- | PMS2_ΔNTD | PMS2 | domain | 1.69 | 42 |
| | PMS2_ΔNTD- | | | truncation | | |
| | NLS(SV40)- | | | | | |
| | mCherry | | | | | |
| pMMR43 | pR- | MLH1co_NTD | MLH1 | domain | 3.18 | 43 |
| | MLH1co_NTD- | | | truncation | | |
| | mCherry | | | | | |
| pMMR44 | pR-NLS(cMyc)- | MLH1co_NTD | MLH1 | domain | 3.36 | 44 |
| | MLH1co_NTD- | | | truncation | | |
| | mCherry | | | | | |
| pMMR45 | pR- | MLH1co_NTD | MLH1 | domain | 3.5 | 45 |
| | MLH1co_NTD- | | | truncation | | |
| | NLS(SV40)- | | | | | |
| | mCherry | | | | | |
| pMMR46 | pR- | MLH1_Δ754- | MLH1 | short | 3.24 | 46 |
| | MLH1_Δ754- | 756 | | deletion | | |
| | 756-mCherry | | | | | |
| pMMR47 | pR-NLS(cMyc)- | MLH1_Δ754- | MLH1 | short | 4.3 | 47 |
| | MLH1_Δ754- | 756 | | deletion | | |
| | 756-mCherry | | | | | |
| pMMR48 | pR- | MLH1_Δ754- | MLH1 | short | | 48 |
| | MLH1_Δ754- | 756 | | deletion | | |
| | 756-NLS(SV40)- | | | | | |
| | mCherry | | | | | |

The MMR constructs included open reading frames (ORFs) of the MMR proteins, either as wild-type, mutant, or truncation variants, which were tested with or without an NLS at either the N′ end (a cMyc sequence) or the C′ end (an SV40 sequence) end of the ORF. The position of the NLS is reflected in the “description” of the construct in Table 1. Table 1 further includes each construct “variant type,” base variant, base gene, as well as the amino acid SEQ ID NO of the mature protein product for each MMR construct (including NLS sequences). In this experiment, mCherry was expressed as a ribosomal-skip (T2A) tag and thus, the mature protein product (and AA sequence) for each MMR construct did not comprise an mCherry tag.

Controls for this experiment included cells transformed with a first editing plasmid and a second “control MMR expression plasmid” comprising no MMR protein sequence, as well as cells transformed with a first editing plasmid and a second plasmid encoding a small ubiquitin-related modifier 4 (SUMO4) protein.

96 hours post transfection (96 hpt) with the vectors, the IPSCs were assayed via a flow cytometry and edit rates (as % BFP+) calculated. Table 1 above includes the fold-improvement in editing performance for the experimental samples transformed with MMR constructs over control samples, averaged for all CFgRNAs. As shown, average fold-improvement ranged from −0.94× (little to no improvement, on average) to ˜4.3× (a significant improvement, on average), with a most MMR constructs facilitating at least some improvement in editing performance.

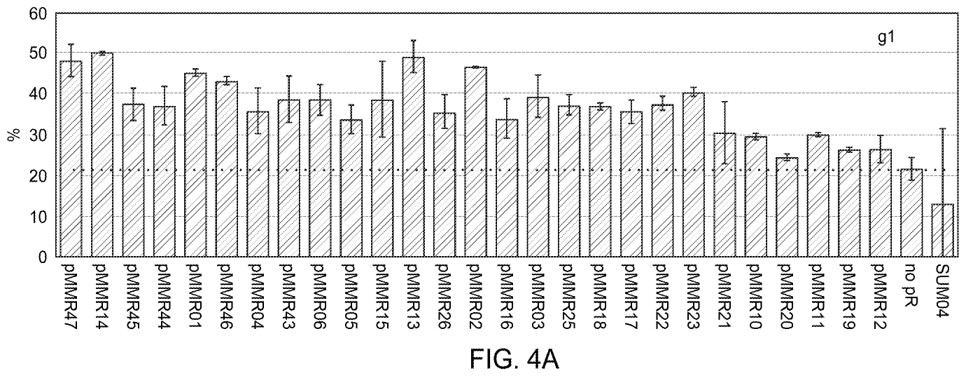

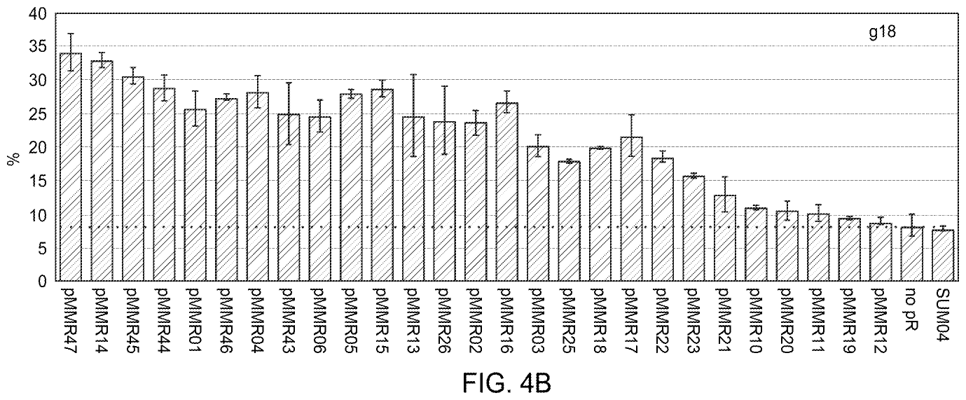

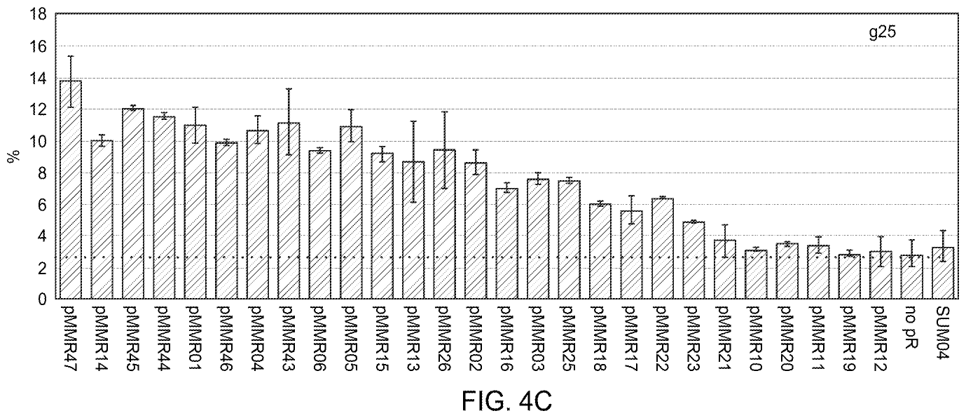

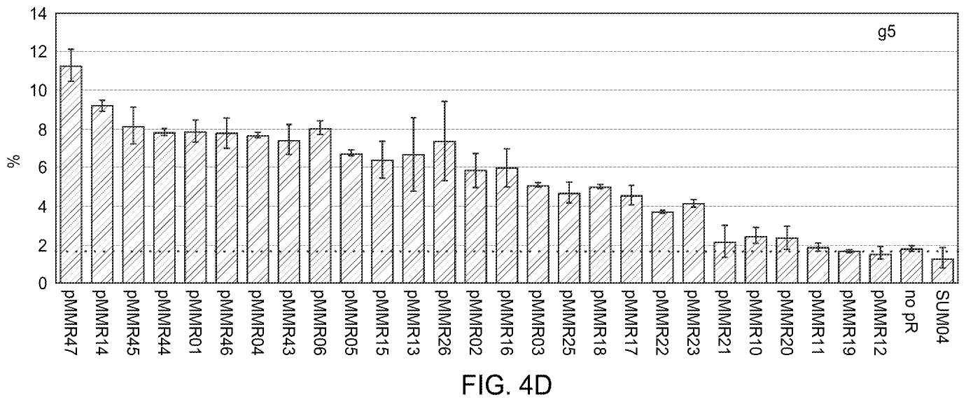

Additionally, FIGS. 4A-4D graphically illustrate the edit rates (as % BFP+) for each of the four CFgRNAs with several MMR constructs from Table 1 that facilitated improved editing performance as compared to baseline: FIG. 4A illustrates the % edit rates of g1 with such MMR constructs; FIG. 4B illustrates the % edit rates of g18 with such MMR constructs; FIG. 4C illustrates the edit rates of g25 with such MMR constructs; and FIG. 4D illustrates the % edit rates of g5 with such MMR constructs. As shown, edit rates were significantly improved for all four CFgRNAs by most of the selected MMR constructs. In many instances, the edit rate was increased by ˜5%, ˜10%, ˜15%, ˜20%, ˜25%, or more.

Upon analysis of the results, it was found that the primary component of the MutL-homolog complex, MLH1, and the primary component of the MutS-homolog complex, MSH2, facilitated, on average, the largest improvement in editing performance for all four GFP-to-BFP CFgRNAs as either wild-type or mutant variants, with wtMLH1 being a top performer. These results suggest that MLH1 and MSH2 are more sensitive to MMR perturbation as compared to other MMR proteins because they are key components of their respective MMR complexes. Additionally, though expression of wild-type, truncated, and mutant variants of such MMR proteins were shown to significantly improve editing performance, transient overexpression of wild-type MMR proteins may provide the most substantial improvement.

### Example V: CREATE Fusion Editing Enhanced by Overexpression of MMR Proteins

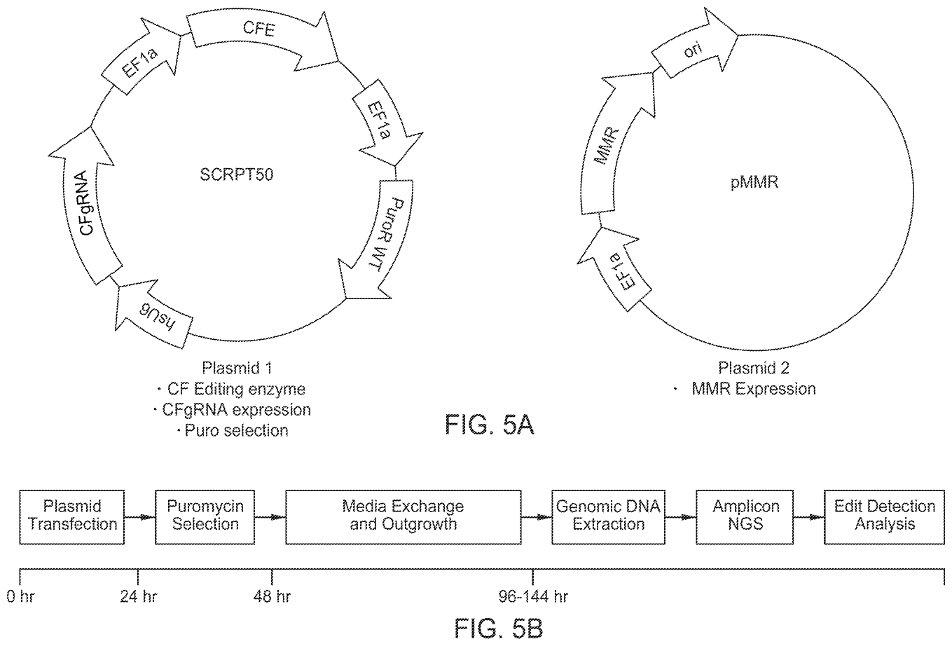

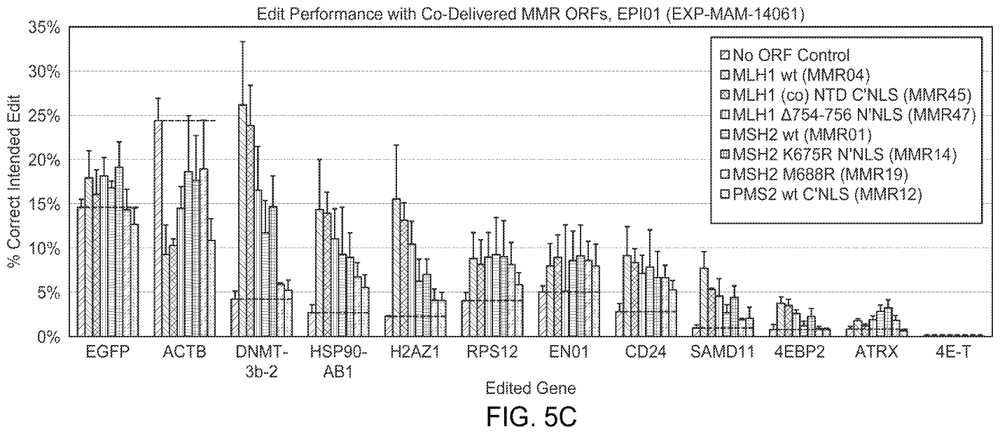

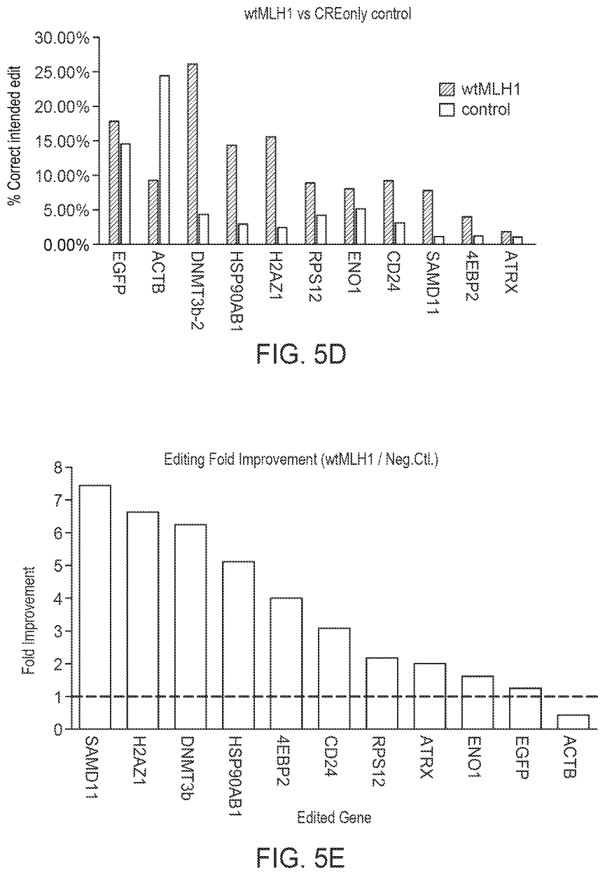

CREATE fusion editing was again carried out in iPSCs using a two-vector system, wherein the two-vector system included a first editing plasmid comprising a nucleotide sequence encoding a CFgRNA and corresponding CF enzyme, and a second MMR expression plasmid comprising an ORF sequence of one of seven experimental MMR proteins. The design of the two plasmids is illustrated in FIG. 5A.

As shown, the “editing” plasmid (here, “SCRPT50”) comprised: a nucleotide sequence encoding a CFgRNA under the transcriptional control of a hsU6 promoter and having a repair template comprising an intended edit (either an insertion, swap, or deletion edit type) for at least one of twelve (12) targeted genomic loci; a nucleotide sequence encoding a corresponding CF enzyme (depicted as “CFE”) under the control of a first EF1α promoter; and, a nucleotide sequence encoding a PuroR wild-type antibiotic resistance marker under the control of a second EF1α promoter. The target locus, edit type, nick-to-edit length (in bases), and edit length (in bases) for each experimental CFgRNA are shown below in Table 2.

TABLE 2

| Design | Target Locus | Edit Type | Nick-to-Edit | Edit Length |
|---|---|---|---|---|
| 1 | ACTB | Insertion | 1 | 3 |
| 2 | DNMT3b-2 | Swap | 1 | 25 |
| 3 | CD24 | Swap | 2 | 4 |
| 4 | HSP90AB1 | Swap | 3 | 4 |
| 5 | ENO1 | Swap | 5 | 2 |
| 6 | RPS12 | Swap | 2 | 4 |
| 7 | H2AZ1 | Swap | 2 | 4 |
| 8 | SAMD11 | Insertion | 1 | 3 |
| 9 | ATRX | Insertion | 2 | 3 |
| 10 | 4EBP2 | Deletion | 1 | 3 |
| 11 | EGFP | Swap | X | 5 |
| 12 | 4E-T | ?? | ?? | ?? |

Meanwhile, the “MMR expression” plasmid (here, “pMMR”) comprised: one of seven MMR nucleotide constructs under the transcriptional control of a third EF1α promoter and comprising an ORF sequence encoding the MMR protein (“MMR”); and, an origin of replication (depicted as “ORI”). For controls, an MMR expression plasmid with no MMR construct/ORF was used.

The seven tested MMR constructs included top and bottom performers of a previous screen of dozens of MMR-derived genes in a GFP-to-BFP edit experiment (including wild-type, point mutants, domain deletions, dominant-negatives, variable NLS tags, etc., as described in Example IV above and found in Table 1). Such top and bottom performers were: pMMR04, pMMR45, pMMR47, pMMR01, pMMR14, pMMR19, and pMMR12. Again, the MMR constructs included the MMR protein ORFs, either as wild-type, mutant, or truncation variants, which were tested with or without an NLS. Position of the NLS is reflected in the “construct description” column in Table 1. Table 1 further lists each construct variant type, as well as the amino acid SEQ ID NO of the mature protein product for each MMR construct (including NLS sequences). mCherry was expressed as a ribosomal-skip (T2A) tag and thus, the mature protein product (and AA sequence) for each MMR construct did not comprise an mCherry tag.

Briefly, PGP168-GFP cells were cultured in mTeSR Plus medium (Stemcell Technologies, Vancouver, CA) at 37° C. and 5% CO₂. 24 hours before transfection with vectors, the cells were seeded at 15,000 cells per well in Matrigel-coated (Corning, NY, USA) flat bottom 96-well culture plates and supplemented with 10 μM Y-27632 ROCK inhibitor (Stemcell Technologies). 100 μL of medium was replaced (without Y-27632) immediately before transfection.

The plasmids were prepared by mixing 50 ng of SCRPT50 plasmid and 50 ng of pMMR plasmid (100 ng of total DNA) with 0.75 μL Lipofectamine Stem Transfection Reagent (Thermo Fisher Scientific, MA, USA) in 10 μL of OptiMEM Reduced Serum Media (Thermo Fisher Scientific). The resulting mixtures was incubated for 10-30 minutes at room temperature, and then added to a single well of cultured PGP168-GFP cells in the 96-well plates and transferred to a 37° C. incubator with 5% CO₂ for co-transfection of the plasmids. As shown in the exemplary flow diagram of FIG. 5B, the transfection medium was removed after 24 hours of incubation and replaced with mTeSR Plus medium (Stemcell Technologies) and 10 μg/mL puromycin (Invivogen, CA, USA) for enrichment of transfected cells. 48 hours after transfection, the medium was again replaced with mTeSR Plus (Stemcell Technologies).

96 hours to 144 hours after transfection, genomic DNA was purified from the cells using a Beckman Coulter DNAdvance gDNA extraction kit (Brea, CA, US), and PCR was performed to amplify regions of genes containing the target sites of the CFgRNAs expressed from the SCRPT50 plasmid. The PCR amplicons were prepared for next-generation sequencing (NGS) using an Illumina TruSeq DNA Sample Preparation Kit according to the manufacturer's instructions, and the samples were sequenced using an Illumina MiSeq using the 2×150 Reagent Kit (Illumina, San Diego, CA, USA).

NGS analysis was performed using a custom read alignment pipeline to bin read counts according to sequence identity to target genomic loci with a complete precise target genomic edit or a wild-type sequence. Precise edit rates of samples with and without each of the MMR constructs at the 12 endogenous target genomic loci were then calculated as a fraction of the total reads aligned to the target genomic locus that contained the precise edit sequence, with the results shown in FIG. 5C.

In FIG. 5C, the precise edit rates (% correct intended edit) at each target genomic locus are shown for experimental samples wherein CFgRNAs were co-delivered and co-expressed with an MMR protein (as a construct), as well as for control samples wherein editing was carried out without MMR protein co-expression (control plasmid). As shown, for most of the target genomic loci tested, the occurrence of precise editing increased between ˜5% and ˜30% when the CFgRNAs were co-delivered and co-expressed with an MMR protein. There was only one CFgRNA design, “Design 1” in Table 2 (ACTB), which generally showed reduced editing performance with MMR protein overexpression. The top performers from the MMR proteins utilized in this experiment included wtMLH1 and dnMLH1s, though transient overexpression of wtMLH1 improved editing significantly more on average than direct MMR inhibition with dnMLH1s. Meanwhile, wtPMS2, though providing some improved editing performance at most of the target genomic loci, was a bottom performer.

Specific results for the top performer, wtMLH1, are shown in FIGS. 5D and 5E. More particularly, FIG. 5D depicts the precise edit rate (% correct intended edits) at 11/12 of the previous target genomic loci for experimental samples wherein the CFgRNA was co-delivered and co-expressed with wtMLH1, as well as for control samples. FIG. 5E, meanwhile, depicts the fold-improvement between the experimental and control samples. Again, for most of the target genomic loci tested, precise editing significantly improved when the with transient overexpression of exogenous wtMLH1. For a majority of the target genomic loci shown (6/11), the incidence of precise editing improved 2-fold, 3-fold, or more with the overexpression of wtMLH1 as compared to the negative control, such that there was a 3.6× average fold-improvement across the experimental constructs between the control samples and experimental samples. Thus, contrary to the recent literature reporting that MMR inhibition improves editing, these results show that transient overexpression of wild-type MMR proteins (or other polypeptides) during editing facilitates substantial edit performance improvement at various endogenous loci, even for different types of edits. Even further, such results suggest that overexpression of wild-type MMR proteins may perturb cellular MMR mechanisms, which may otherwise hamper editing performance.

### Example VI: CREATE Fusion Editing with Simultaneous Overexpression of Multiple MMR Proteins

CREATE fusion editing was also carried out in iPSCs to determine the effects of co-expressing a combination of MMR proteins along with CF editing machinery, as compared to expression of individual MMR proteins or no MMR proteins. Again, a two-vector system was used. The two vectors included a first editing plasmid comprising a nucleotide sequence encoding one of 96 CFgRNAs designed to test diversity in edit type, nick-to-edit distance, and observed edit efficiency, as well as a corresponding CF enzyme, and a second MMR expression plasmid comprising one or two nucleotide sequences encoding MMR proteins.

A similar plate editing workflow as described in Example V was performed. This time, four different conditions were tested, which are listed in Table 3 below. The conditions included: no delivery/expression of MMR proteins (control) during editing; co-delivery/co-expression of wild-type MLH1; co-delivery/co-expression of dominant-negative mutant MSH2; and co-delivery/co-expression of both wild-type MLH1 and dominant-negative mutant MSH2. Each MMR protein was delivered as an MMR nucleotide construct with or without a nuclear localization signal. Similar to Table 1 above, Table 3 includes information relating to the NLS, variant type, and mature AA SEQ ID NO for each construct. Again, mCherry was expressed as a ribosomal-skip (T2A) tag and thus, the mature protein product (and AA sequence) for each MMR construct did not comprise an mCherry tag.

TABLE 3

| | MMR | MMR Con- | | AA |
|---|---|---|---|---|
| | Con- | struct | Variant | SEQ |
| Condition | struct(s) | Full Name(s) | Type | ID NO |
| Control | None | None | pUC-FC02 | N/A |
| | | | Plasmid | |
| MLH1wt | pMMR04 | pR-wtMLH1-mCherry | Wild-type | 04 |
| MSH2mut | pMMR14 | pR-NLS(cMyc)- | Point mutant | 14 |
| | | MSH2_K675R- | | |
| | | mCherry | | |
| MLH1wt + | pMMR04, | pR-wtMLH1-mCherry, | Combination | 04, 14 |
| MSH2mut | pMMR14 | pR-NLS(cMyc)- | wild-type | |
| | | MSH2_K675R- | and point | |
| | | mCherry | mutant | |

Note that at no point during the editing workflow was there any indication that transient overexpression of MLH1wt and/or MSH2 disrupted either iPSC cell morphology or the ability of the cells to differentiate into three germ layers.

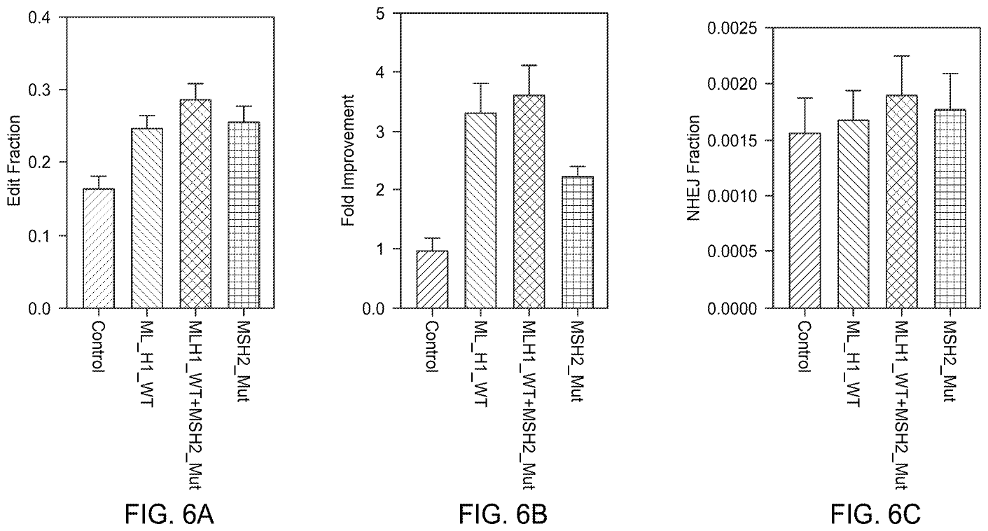

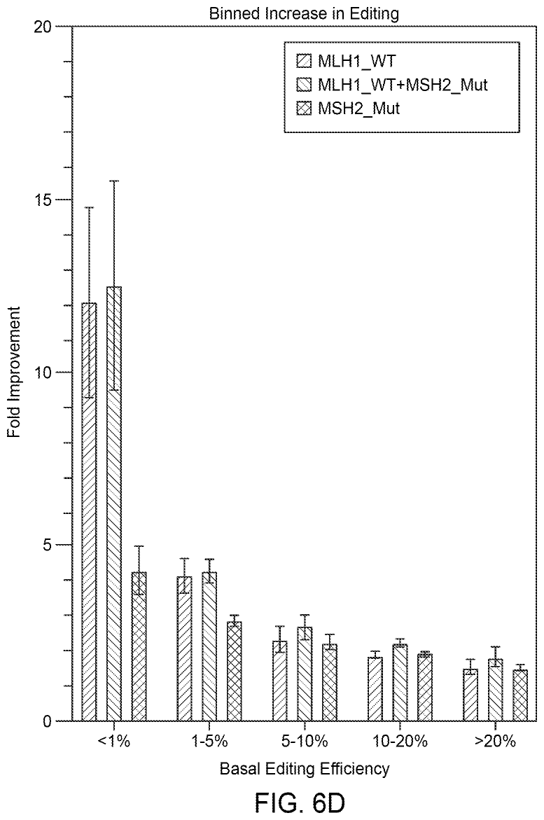

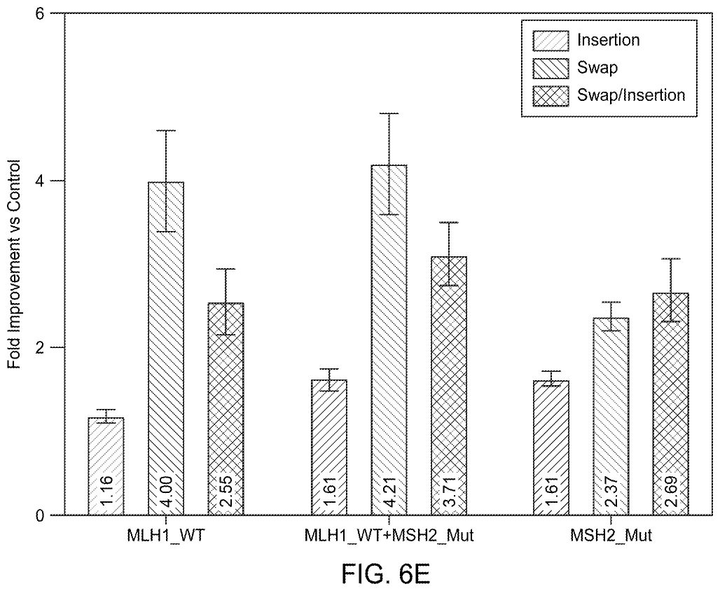

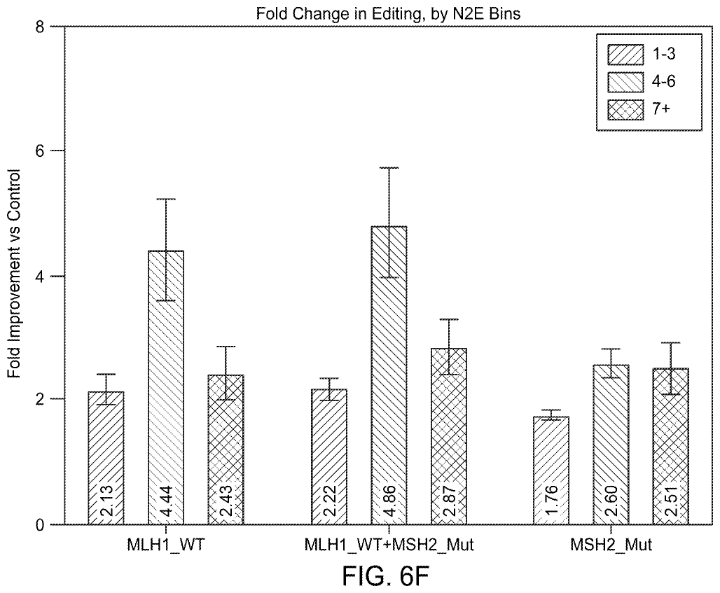

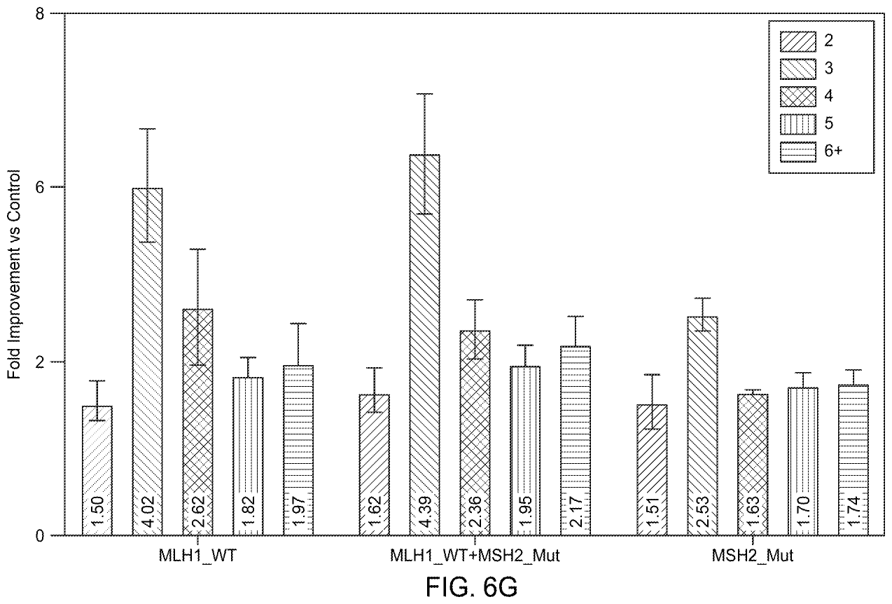

NGS analysis was performed using the custom read alignment pipeline described above and edit rates for experimental and control samples were calculated as a fraction of total reads aligned to the target genomic locus that contained the correct edit sequence, with the results shown in FIGS. 6A and 6B. In FIG. 6A, the on-target edit rates (correct intended “edit fraction”) for each condition are shown averaged across all genomic targets. Meanwhile, FIG. 6B depicts the average fold-improvement between the experimental and control samples. It was found that the MLH1wt and MSH2mut ORFs each behaved differently for different target genomic loci and different CFgRNA designs. Yet, individual expression of MLH1wt or MSH2mut during editing yielded a ˜1.6× average improvement in correct intended edit (CIE) rate as compared to no MMR protein expression, with an average edit performance improvement (EPI) of about ˜3× or ˜2×, respectively. Meanwhile, co-expression of both MLH1wt and MSH2mut yielded a ˜2× improvement in CIE rate as compared to no MMR protein expression, with an average EPI of about ˜3.5×. Accordingly, the results suggest that transient overexpression of both MLH1wt and MSH2mut provided a partially additive effect on editing performance, and thus, at genomic target sites where, e.g., MLH1wt overexpression did not improve or decrease editing performance, MSH2mut overexpression may have shown a compensatory higher editing performance improvement, and vice versa. Therefore, transient overexpression of two or more different MMR proteins (or other MMR polypeptides) during editing may yield greater editing performance improvement as compared to overexpression of a single MMR protein.

In addition to on-target edit rates, the rates of non-homologous end joining (NHEJ) were also determined for all four conditions. FIG. 6C depicts the occurrence of NHEJ (NHEJ fraction) for each condition averaged across all genomic targets. As shown, the individual and dual expression of MLH1wt and MSH2mut during editing of the experimental samples resulted in only a nominal increase in NHEJ as compared to controls. Accordingly, these results suggest that MMR disruption via expression/overexpression of MMR proteins (or other polypeptides) induces a robust improvement in targeted editing performance without substantially increasing NHEJ.

Analysis was also performed using the custom read alignment pipeline to determine editing performance improvement of the conditions with MLH1wt and/or MSH2mut at target genomic loci having varying basal editing efficiencies. FIG. 6D illustrates the binned increase in editing with co-expression of MLH1wt and/or MSH2mut at target loci having basal editing efficiencies of <1%, 1-5%, 5-10%, 10-20%, and >20%. As shown, transient overexpression of MSH1wt, and particularly, MSH1wt in combination with MSH2mut, predominantly improved editing at very difficult targets (lower basal editing efficiencies of, e.g., <1% and 1-5%).

The editing results for conditions with MLH1wt and/or MSH2mut were further parsed based on the edit type, nick-to-edit distance, and edit length of the 96 CFgRNAs assembled into the editing plasmid of the two-vector system. FIG. 6E illustrates the average fold-improvement (vs control) of each of the MLH1wt and/or MSH2mut conditions for each edit type of the tested CFgRNAs (insertion, swap, or swap/insertion); FIG. 6F illustrates the average fold-improvement (vs control) of each of the MLH1wt and/or MSH2mut conditions based on nick-to-edit distance of the tested CFgRNAs (1-3 bases, 4-6 bases, or 7+ bases); FIG. 6G illustrates the average fold-improvement (vs control) of each of the MLH1wt and/or MSH2mut conditions based on edit length of the tested CFgRNAs (2, 3, 4, 5, or 6+ bp). As shown in FIGS. 6E and 6F, CFgRNAs with medium-length (4-6 bases) nick-to-edit distances and swap edits showed the most performance improvement as a result of MMR protein expression. Meanwhile, in FIG. 6G, CFgRNAs with edits of 3-4 bp showed the most editing performance improvement with expression of MLH1wt and/or MSH2mut.

In furtherance of the combinatorial testing of MLH1wt and/or MSH2mut using the aforementioned MMR constructs (pMMR01, pMMR14), additional combinations of MMR constructs from Table 1 were screened against CFgRNAs targeting 12 genomic loci for effect on editing performance. These combinations included: pMMR47 and pMMR14; pMMR14 and pMMR30; pMMR 14 and pMMR47 and pMMR30; pMMR01 and pMMR04; pMMR01 and pMMR07; and pMMR01 and pMMR04 and pMMR07, which are listed in Table 4 below. Table 4 further includes the fold-improvement between experimental and control samples averaged across all the genomic targets.

TABLE 4

| | Average Fold- |
|---|---|
| Combination | Improvement |
| pMMR47 + pMMR14 | 2.42x |
| pMMR14 + pMMR30 | 1.71x |
| pMMR14 + pMMR47 + pMMR30 | 1.96x |
| pMMR01 + pMMR04 | 2.24x |
| pMMR01 + pMMR07 | 1.41x |
| pMMR01 + pMMR04 + pMMR07 | 2.14x |

As shown, while all combinations exhibited some improvement in editing performance, those combinations comprising MLH1 exhibited greater improvement than others.

### Example VII: Screening Additional Genes for Enhanced CREATE Fusion Editing

CREATE fusion editing was carried out in iPSCs to identify additional genes that, when expressed during CF editing alone or in combination with, e.g., MMR proteins, would improve precise edit rates. Similar to the workflow in Example IV, the genes were screened against four different CFgRNAs encoding a GFP-to-BFP edit to see if expression/overexpression thereof would improve editing performance. Thirty-nine (39) different genes, delivered as nucleotide constructs to the IPSCs in a two-vector system, were designed and/or tested. The constructs are listed below in Table 5.

TABLE 5

| | | | | | Avg Fold- |
|---|---|---|---|---|---|
| Construct | | | Base | Variant | Improvement |
| No. | Construct description | Base variant | Gene | Type | Over Control |
| pMMR49 | pR-MEAF6-mCherry | wtMEAF6 | MEAF6 | wild-type | 1.1 |
| | | | | ORF | |
| pMMR50 | pR-NLS(cMyc)-MEAF6- | wtMEAF6 | MEAF6 | wild-type | 0.88 |
| | mCherry | | | ORF | |
| pMMR51 | pR-MEAF6-NLS(SV40)- | wtMEAF6 | MEAF6 | wild-type | |
| | mCherry | | | ORF | |
| pMMR52 | pR-MPG-mCherry | wtMGG | MPG | wild-type | 1.23 |
| | | | | ORF | |
| pMMR53 | pR-NLS(cMyc)-MPG-mCherry | wtMPG | MPG | wild-type | |
| | | | | ORF | |
| pMMR54 | pR-MPG-NLS(SV40)-mCherry | wtMPG | MPG | wild-type | 1.44 |
| | | | | ORF | |
| pMMR55 | pR-LIG1-mCherry | wtLIG1 | LIG1 | wild-type | 0.89 |
| | | | | ORF | |
| pMMR56 | pR-NLS(cMyc)-LIG1-mCherry | wtLIG1 | LIG1 | wild-type | 0.9 |
| | | | | ORF | |
| pMMR57 | pR-LIG1-NLS(SV40)-mCherry | wtLIG1 | LIG1 | wild-type | 1 |
| | | | | ORF | |
| pMMR58 | pR-LIG3_A9-mCherry | wtLIG3 | LIG3 | wild-type | 1.27 |
| | | (A9) | (A9) | ORF | |
| pMMR59 | pR-NLS(cMyc)-LIG3_A9- | wtLIG3 | LIG3 | wild-type | 0.96 |
| | mCherry | (A9) | (A9) | ORF | |
| pMMR60 | pR-LIG3_A9-NLS(SV40)- | wtLIG3 | LIG3 | wild-type | 1.13 |
| | mCherry | (A9) | (A9) | ORF | |
| pMMR61 | pR-LIG3_B5-mCherry | wtLIG3 | LIG3 | wild-type | 1.49 |
| | | (B5) | (B5) | ORF | |
| pMMR62 | pR-NLS(cMyc)-LIG3_B5- | wtLIG3 | LIG3 | wild-type | |
| | mCherry | (B5) | (B5) | ORF | |
| pMMR63 | pR-LIG3_B5-NLS(SV40)- | wtLIG3 | LIG3 | wild-type | 1.1 |
| | mCherry | (B5) | (B5) | ORF | |
| pMMR64 | pR-FEN1-mCherry | wtFEN1 | FEN1 | wild-type | 1.1 |
| | | | | ORF | |
| pMMR65 | pR-NLS(cMyc)-FEN1- | wtFEN1 | FEN1 | wild-type | |
| | mCherry | | | ORF | |
| pMMR66 | pR-FEN1-NLS(SV40)- | wtFEN1 | FEN1 | wild-type | 1.1 |
| | mCherry | | | ORF | |
| pMMR67 | pR-MRGBP-mCherry | wtMRGBP | MRGBP | wild-type | 1.29 |
| | | | | ORF | |
| pMMR68 | pR-NLS(cMyc)-MRGBP- | wtMRGBP | MRGBP | wild-type | 1.2 |
| | mCherry | | | ORF | |
| pMMR69 | pR-MRGBP-NLS(SV40)- | wtMRGBP | MRGBP | wild-type | 1.18 |
| | mCherry | | | ORF | |
| pMMR70 | pR-KAT7-mCherry | wtKAT7 | KAT7 | wild-type | 1.26 |
| | | | | ORF | |
| pMMR71 | pR-NLS(cMyc)-KAT7- | wtKAT7 | KAT7 | wild-type | 1.21 |
| | mCherry | | | ORF | |
| pMMR72 | pR-KAT7-NLS(SV40)- | wtKAT7 | KAT7 | wild-type | |
| | mCherry | | | ORF | |
| pMMR73 | pR-RPA2-mCherry | wtRPA2 | RPA2 | wild-type | |
| | | | | ORF | |
| pMMR74 | pR-NLS(cMyc)-RPA2- | wtRPA2 | RPA2 | wild-type | 1.17 |
| | mCherry | | | ORF | |
| pMMR75 | pR-RPA2-NLS(SV40)- | wtRPA2 | RPA2 | wild-type | 1.22 |
| | mCherry | | | ORF | |
| pMMR76 | pR-CHAF1B-mCherry | wtCHAF1B | CHAF1B | wild-type | 1.23 |
| | | | | ORF | |
| pMMR77 | pR-NLS(cMyc)-CHAF1B- | wtCHAF1B | CHAF1B | wild-type | |
| | mCherry | | | ORF | |
| pMMR78 | pR-CHAF1B-NLS(SV40)- | wtCHAF1B | CHAF1B | wild-type | 1.21 |
| | mCherry | | | ORF | |
| pMMR79 | pR-ALKBHZ-mCherry | wtALKBHZ | ALKBHZ | wild-type | |
| | | | | ORF | |
| pMMR80 | pR-NLS(cMyc)-ALKBHZ- | wtALKBHZ | ALKBHZ | wild-type | |
| | mCherry | | | ORF | |
| pMMR81 | pR-ALKBHZ-NLS(SV40)- | wtALKBHZ | ALKBHZ | wild-type | |
| | mCherry | | | ORF | |
| pMMR82 | pR-NUDT1_B4-mCherry | wtNUDT1 | NUDT1 | wild-type | 1.26 |
| | | (B4) | (B4) | ORF | |
| pMMR83 | pR-NLS(cMyc)-NUDT1_B4- | wtNUDT1 | NUDT1 | wild-type | 0.86 |
| | mCherry | (B4) | (B4) | ORF | |
| pMMR84 | pR-NUDT1_B4-NLS(SV40)- | wtNUDT1 | NUDT1 | wild-type | 1.19 |
| | mCherry | (B4) | (B4) | ORF | |
| pMMR85 | pR-NUDT1_B8-mCherry | wtNUDT1 | NUDT1 | wild-type | 1.38 |
| | | (B8) | (B8) | ORF | |
| pMMR86 | pR-NLS(cMyc)-NUDT1_B8- | wtNUDT1 | NUDT1 | wild-type | |
| | mCherry | (B8) | (B8) | ORF | |
| pMMR87 | pR-NUDT1_B8-NLS(SV40)- | wtNUDT1 | NUDT1 | wild-type | |
| | mCherry | (B8) | (B8) | ORF | |

The constructs included open reading frames (ORFs) of the various genes/proteins as wild-type, which were tested with or without an NLS at either the N′ end or the C′ end of the ORF. The position of the NLS is reflected in the “description” column of Table 5. Table 5 further includes each construct base variant and gene, as well as the average fold-improvement in editing performance over controls.

As shown, average fold-improvement ranged from ˜0.86× (little to no improvement, on average) to ˜1.49× (a significant improvement, on average), with most constructs facilitating at least some improvement in editing performance. Accordingly, the results indicate that co-delivering/co-expressing such constructs along with CF editing machinery can help improve editing performance. Even further, there is the potential to combine such constructs with, e.g., the MMR proteins described above, to facilitate additional editing performance improvement.

While this disclosure is satisfied by embodiments in many different forms, as described in detail in connection with preferred embodiments of the disclosure, it is understood that the present disclosure is to be considered as exemplary of the principles of the disclosure and is not intended to limit the disclosure to the specific embodiments illustrated and described herein. Numerous variations may be made by persons skilled in the art without departure from the spirit of the disclosure. The scope of the disclosure will be measured by the appended claims and their equivalents. The abstract and the title are snot to be construed as limiting the scope of the present disclosure, as their purpose is to enable the appropriate authorities, as well as the general public, to quickly determine the general nature of the disclosure. In the claims that follow, unless the term “means” is used, none of the features or elements recited therein should be construed as means-plus-function limitations pursuant to 35 U.S.C. § 112, ¶6.

### EXAMPLE EMBODIMENTS

Embodiment 1: A system for nucleic acid-guided editing in a genome of a cell, comprising: a first polynucleotide sequence encoding an editing cassette, the editing cassette comprising an edit to a target genomic locus of a cell; a second polynucleotide sequence encoding at least a portion of a first DNA repair protein participating in a DNA repair mechanism of the cell; and a third polynucleotide sequence encoding a nucleic acid-guided nuclease or a nickase-RT fusion.

Embodiment 2: The system of Embodiment 1, wherein the first DNA repair protein comprises a protein participating in a DNA break response mechanism.

Embodiment 3: The system of Embodiment 1, wherein the first DNA repair protein comprises a protein participating in a base excision repair (BER) mechanism, a nucleotide excision repair (NER) mechanism, a homologous recombination (HR) mechanism, or a non-homologous end joining (NHEJ) mechanism of the cell.

Embodiment 4: The system of Embodiment 1, wherein the second polynucleotide sequence encodes an open reading frame (ORF) of the first DNA repair protein.

Embodiment 5: The system of Embodiment 1, wherein the first DNA repair protein comprises a mismatch repair (MMR) protein.

Embodiment 6: The system of Embodiment 5, wherein the first DNA repair protein comprises a wild-type MMR protein of the cell.

Embodiment 7: The system of Embodiment 5, wherein the first DNA repair protein comprises a mutant or modified MMR protein.

Embodiment 8: The system of Embodiment 6, wherein the first DNA repair protein comprises a wild-type MLH1 protein, a wild-type MSH2 protein, a wild-type MSH6 protein, a wild-type PMS2 protein, or a wild-type component of a MutSα-MutLα MMR complex.

Embodiment 9: The system of Embodiment 7, wherein the first DNA repair protein comprises a mutant MLH1 protein, a mutant MSH2 protein, a mutant MSH6 protein, a mutant PMS2 protein, or a mutant component of a MutSα-MutLα MMR complex.

Embodiment 10: The system of Embodiment 1, wherein the first DNA repair protein encoded by the second polynucleotide sequence is under the control of an inducible promoter.

Embodiment 11: The system of Embodiment 10, wherein the first DNA repair protein encoded by the second polynucleotide sequence is under the control of an inducible promoter that is separate and different than an inducible promoter controlling the editing cassette.

Embodiment 12: The system of Embodiment 1, wherein the first DNA repair protein encoded by the second polynucleotide sequence is under the control of a constitutive promoter.

Embodiment 13: The system of Embodiment 1, further comprising: a fourth polynucleotide sequence encoding at least a portion of a second DNA repair protein.

Embodiment 14: The system of Embodiment 13, wherein the fourth polynucleotide sequence encodes an open reading frame (ORF) of the second DNA repair protein.

Embodiment 15: The system of Embodiment 13, wherein the second DNA repair protein comprises a wild-type MMR protein of the cell.

Embodiment 16: The system of Embodiment 13, wherein the second DNA repair protein comprises a mutant or modified MMR protein.

Embodiment 17: The system of Embodiment 16, wherein the second DNA repair protein comprises a dominant negative MMR protein that is catalytically impaired.

Embodiment 18: The system of Embodiment 1, wherein the first and second DNA repair proteins are different DNA repair proteins.

Embodiment 19: The system of Embodiment 18, wherein the first DNA repair protein is a functional DNA repair protein, and wherein the second DNA repair protein is a catalytically impaired DNA repair protein.

Embodiment 20: The system of Embodiment 18, wherein the first and second DNA repair proteins are different MMR proteins.

Embodiment 21: The system of Embodiment 20, wherein the first DNA repair protein is a functional MMR protein, and wherein the second DNA repair protein is a catalytically impaired MMR protein.

Embodiment 22: The system of Embodiment 13, wherein the second DNA repair protein encoded by the fourth polynucleotide sequence is under the control of an inducible promoter.

Embodiment 23: The system of Embodiment 13, wherein second DNA repair protein encoded by the fourth polynucleotide sequence is under the control of an inducible promoter that is separate and different than an inducible promoter controlling the editing cassette or an inducible promoter controlling the first DNA repair protein encoded by the second polynucleotide sequence.

Embodiment 24: The system of Embodiment 13, wherein the second DNA repair protein encoded by the fourth polynucleotide sequence is under the control of a constitutive promoter.

Embodiment 25: The system of Embodiment 1, wherein the first polynucleotide sequence, the second polynucleotide sequence, and the third polynucleotide sequence are comprised on one or more vector constructs.

Embodiment 26: The system of Embodiment 25, wherein second polynucleotide sequence is comprised on a vector construct different from a vector construct of the first polynucleotide sequence or the third polynucleotide sequence.

Embodiment 27: The system of Embodiment 1, wherein the first polynucleotide sequence encoding the editing cassette comprises a sequence encoding a gRNA transcript having a region of complementarity to a sequence of the target genomic locus and configured to guide the nuclease to the target genomic locus.

Embodiment 28: The system of Embodiment 1, wherein the first polynucleotide sequence encoding the editing cassette comprises a sequence encoding a repair template transcript comprising the edit.

Embodiment 29: The system of Embodiment 28, wherein the sequence encoding the repair template transcript is covalently linked to a sequence of the first polynucleotide sequence encoding a gRNA transcript.

Embodiment 30: The system of Embodiment 28, wherein the repair template transcript further comprises an immunizing edit to prevent subsequent editing of the target genomic locus.

Embodiment 31: The system of Embodiment 1, wherein the nuclease comprises a MAD nuclease, a MAD nickase, or a variant thereof.

Embodiment 32: The system of Embodiment 31, wherein the nuclease comprises one or more of MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, and MAD299.

Embodiment 33: The system of Embodiment 1, wherein the nuclease comprises a Cas9 nuclease, a Cas9 nickase, or a variant thereof.

Embodiment 34: The system of Embodiment 1, wherein the nuclease comprises one or more of C2c1, C2c2, C2c3, Cas1, Cas1B, Cas2, Cas3, Cas4, Cas5, Cas6, Cas7, Cas8, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, and Csf4.

Embodiment 35: A system for nucleic acid-guided nickase/reverse transcriptase fusion editing in a genome of a cell, comprising: a first polynucleotide encoding a CFgRNA, the CFgRNA comprising a repair template having an edit to a target genomic locus of a cell; a second polynucleotide encoding at least a portion of a first DNA repair protein participating in a DNA repair mechanism of the cell; and a nucleic acid-guided nickase/reverse transcriptase fusion enzyme or a third polynucleotide encoding the nucleic acid-guided nickase/reverse transcriptase fusion enzyme.

Embodiment 36: The system of Embodiment 35, wherein the first DNA repair protein comprises a protein configured to function in a DNA break response mechanism.

Embodiment 37: The system of Embodiment 35, wherein the first DNA repair protein comprises a protein participating in a base excision repair (BER) mechanism, a nucleotide excision repair (NER) mechanism, a homologous recombination (HR) mechanism, or a non-homologous end joining (NHEJ) mechanism of the cell.

Embodiment 38: The system of Embodiment 35, wherein the second polynucleotide encodes an open reading frame (ORF) of the first DNA repair protein.

Embodiment 39: The system of Embodiment 35, wherein the first DNA repair protein comprises a mismatch repair (MMR) protein participating in an MMR pathway of the cell.

Embodiment 40: The system of Embodiment 39, wherein the first DNA repair protein comprises a wild-type MMR protein of the cell.

Embodiment 41: The system of Embodiment 39, wherein the first DNA repair protein comprises a mutant or modified MMR protein.

Embodiment 42: The system of Embodiment 40, wherein the first DNA repair protein comprises a wild-type MLH1 protein, a wild-type MSH2 protein, a wild-type MSH6 protein, a wild-type PMS2 protein, or a wild-type component of a MutSα-MutLα MMR complex.

Embodiment 43: The system of Embodiment 41, wherein the first DNA repair protein comprises a mutant MLH1 protein, a mutant MSH2 protein, a mutant MSH6 protein, a mutant PMS2 protein, or a mutant component of a MutSα-MutLα MMR complex.

Embodiment 44: The system of Embodiment 35, wherein the first DNA repair protein encoded by the second polynucleotide is under the control of an inducible promoter.

Embodiment 45: The system of Embodiment 44, wherein the first DNA repair protein encoded by the second polynucleotide is under the control of an inducible promoter that is separate and different than an inducible promoter controlling the CFgRNA.

Embodiment 46: The system of Embodiment 35, wherein the first DNA repair protein encoded by the second polynucleotide is under the control of a constitutive promoter.

Embodiment 47: The system of Embodiment 35, further comprising: a fourth polynucleotide encoding at least a portion of a second DNA repair protein.

Embodiment 48 The system of Embodiment 47, wherein the fourth polynucleotide encodes an open reading frame (ORF) of the second DNA repair protein.

Embodiment 49: The system of Embodiment 47, wherein the second DNA repair protein comprises a wild-type MMR protein of the cell.

Embodiment 50: The system of Embodiment 47, wherein the second DNA repair protein comprises a mutant or modified MMR protein.

Embodiment 51: The system of Embodiment 50, wherein the second DNA repair protein comprises a dominant negative MMR protein that is catalytically impaired.

Embodiment 52: The system of Embodiment 35, wherein the first and second DNA repair proteins are different DNA repair proteins.

Embodiment 53: The system of Embodiment 52, wherein the first DNA repair protein is a functional DNA repair protein, and wherein the second DNA repair protein is a catalytically impaired DNA repair protein.

Embodiment 54: The system of Embodiment 52, wherein the first and second DNA repair proteins are different MMR proteins.

Embodiment 55: The system of Embodiment 54, wherein the first DNA repair protein is a functional MMR protein, and wherein the second DNA repair protein is a catalytically impaired MMR protein.

Embodiment 56: The system of Embodiment 47, wherein the second DNA repair protein encoded by the fourth polynucleotide is under the control of an inducible promoter.

Embodiment 57: The system of Embodiment 47, wherein second DNA repair protein encoded by the fourth polynucleotide is under the control of an inducible promoter that is separate and different than an inducible promoter controlling the CFgRNA or an inducible promoter controlling the first DNA repair protein encoded by the second polynucleotide.

Embodiment 58: The system of Embodiment 47, wherein the second DNA repair protein encoded by the fourth polynucleotide is under the control of a constitutive promoter.

Embodiment 59: The system of Embodiment 35, wherein the first polynucleotide, the second polynucleotide, and the third polynucleotide are comprised on one or more vector constructs.

Embodiment 60: The system of Embodiment 35, wherein second polynucleotide sequence is comprised on a vector construct different from a vector construct of the first polynucleotide or the third polynucleotide.

Embodiment 61: The system of Embodiment 35, wherein the CFgRNA comprises from 5′ to 3′ a sequence encoding an editing gRNA transcript covalently linked to a repair template transcript.

Embodiment 62: The system of Embodiment 61, wherein the sequence encoding the repair template transcript comprises from 5′ to 3′ a post-edit homology (PEH) region sequence, an edit sequence, a nick-to-edit region sequence, and a primer binding site (PBS) sequence.

Embodiment 63: The system of Embodiment 61, wherein the CFgRNA is disposed on a CF editing cassette further comprising a sequence encoding an RNA G-quadruplex region at a ′3 end of the repair template transcript.

Embodiment 64: The system of Embodiment 35, wherein a nickase portion of the nucleic acid-guided nickase/reverse transcriptase fusion enzyme comprises a MAD nickase or a variant thereof.

Embodiment 65: The system of Embodiment 64, wherein the nickase portion of the nucleic acid-guided nickase/reverse transcriptase fusion enzyme comprises one or more of MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, and MAD299.

Embodiment 66: The system of Embodiment 35, wherein a nickase portion of the nucleic acid-guided nickase/reverse transcriptase fusion enzyme comprises a Cas9 nickase or a variant thereof.

Embodiment 67: The system of Embodiment 35, wherein a nickase portion of the nucleic acid-guided nickase/reverse transcriptase fusion enzyme comprises one or more of C2c1, C2c2, C2c3, Cas1, Cas1B, Cas2, Cas3, Cas4, Cas5, Cas6, Cas7, Cas8, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, and Csf4.

Embodiment 68: A cell comprising any of the systems of Embodiments 1-67.

1. A system for nucleic acid-guided editing in a genome of a cell, the system comprising:
a CREATE fusion guide RNA (CFgRNA) or a polynucleotide sequence encoding the CFgRNA, the CFgRNA comprising an edit to be incorporated into a target genomic locus of the cell;
a nickase-reverse transcriptase (RT) fusion enzyme or a polynucleotide sequence encoding the nickase-RT fusion enzyme; and
a mismatch repair (MMR) perturbation agent comprising a first MMR polypeptide or a polynucleotide sequence encoding the first MMR polypeptide, wherein the first MMR polypeptide comprises a wild-type MMR polypeptide from the cell or another species.

2. The system of claim 1, wherein the MMR perturbation agent further comprises a second MMR polypeptide or a polynucleotide sequence encoding the second MMR polypeptide, wherein the second MMR polypeptide comprises an MMR polypeptide mutant variant.

3. The system of claim 2, wherein the first or the second MMR polypeptide comprises at least one of MLH1, MLH1co, MLH2, MLH3, MSH2, MSH3, MSH6, PMS1, and PMS2.

4. (canceled)

5. The system of claim 2, wherein the second MMR polypeptide comprises a dominant-negative mutant variant, a point mutation mutant variant, a domain truncation mutant variant, or a deletion mutant variant.

6. (canceled)

7. (canceled)

8. (canceled)

9. The system of claim 2, wherein the second MMR polypeptide comprises a K675R mutant variant of MSH2, a K675A mutant variant of MSH2, an M688R mutant variant of MSH2, or a ΔCTD mutant variant of MSH2.

10. (canceled)

11. (canceled)

12. (canceled)

13. (canceled)

14. The system of claim 1, wherein the first MMR polypeptide comprises a mammalian MMR polypeptide or a bacterial MMR polypeptide.

15. (canceled)

16. (canceled)

17. (canceled)

18. (canceled)

19. (canceled)

20. (canceled)

21. The system of claim 2, wherein the first or the second MMR polypeptide comprises an MMR protein complex or a portion of an MMR protein complex.

22. The system of claim 2, wherein the first or the second MMR polypeptide comprises a MutL or MutS protein complex or a portion of a MutL or MutS protein complex.

23. (canceled)

24. (canceled)

25. The system of claim 2, wherein the MMR perturbation agent comprises the polynucleotide sequence encoding the first MMR polypeptide, and wherein the first MMR polypeptide is under the transcriptional control of an inducible promoter; and/or wherein the MMR perturbation agent comprises the polynucleotide sequence encoding the second MMR polypeptide, and wherein the second MMR polypeptide is under the transcriptional control of an inducible promoter.

26. (canceled)

27. The system of claim 1, wherein the CFgRNA comprises:
an editing guide RNA (gRNA) having a region of complementarity to a sequence of the target genomic locus of the cell; and
a repair template covalently linked to the editing gRNA, the repair template comprising the edit to be incorporated into the target genomic locus of the cell.

28. The system of claim 1, wherein the nickase-RT fusion enzyme comprises at least a portion of a MAD-series nickase or variant thereof fused to at least a portion of a reverse transcriptase.

29. The system of claim 28, wherein the MAD-series nickase comprises a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, or MAD299 nickase.

30. The system of claim 1, wherein a nickase portion of the nickase-RT fusion enzyme comprises at least a portion of a Cas9, C2c1, C2c2, C2c3, Cas1, Cas1B, Cas2, Cas3, Cas4, Cas5, Cas6, Cas7, Cas8, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, or Csf4 nickase.

31. A cell comprising the system of claim 1.

32. A method for performing nucleic acid-guided editing in a genome of a cell, the method comprising:
(a) introducing into a cell a system comprising:
a CREATE fusion gRNA (CFgRNA) or a polynucleotide sequence encoding the CFgRNA, the CFgRNA comprising an edit to be incorporated into a target genomic locus of the cell;
a nickase-reverse transcriptase (RT) fusion enzyme or a polynucleotide sequence encoding the nickase-RT fusion enzyme; and
a mismatch repair (MMR) perturbation agent comprising a first MMR polypeptide or a polynucleotide sequence encoding the first MMR polypeptide, wherein the first MMR polypeptide comprises a wild-type MMR polypeptide from the cell or another species; and
(b) providing conditions to allow the CFgRNA and the nickase-RT fusion enzyme to bind to and edit the target genomic locus of the cell while the first MMR polypeptide is expressed.

33. A system for nucleic acid-guided editing in a genome of a cell, the system comprising:
a CREATE fusion gRNA (CFgRNA) or a polynucleotide sequence encoding the CFgRNA, the CFgRNA comprising an edit to be incorporated into a target genomic locus of the cell;
a nickase-reverse transcriptase (RT) fusion enzyme or a polynucleotide sequence encoding the nickase-RT fusion enzyme; and
a mismatch repair (MMR) perturbation agent comprising a first MMR polypeptide or a polynucleotide sequence encoding the first MMR polypeptide, wherein the first MMR polypeptide comprises a wild-type MLH1 from the cell or another species, and wherein the MMR perturbation agent further comprises a second MMR polypeptide comprising a K675R variant of MSH2 or a polynucleotide sequence encoding the second MMR polypeptide.

34. A cell comprising the system of claim 33.

35. A method for performing nucleic acid-guided editing in a genome of a cell, the method comprising:
introducing into the cell the system of claim 33; and
providing conditions to allow the CFgRNA and the nickase-RT fusion enzyme to bind to and edit the target genomic locus of the cell.

36. The method of claim 35, wherein the method achieves at least a 1.5 fold increase in editing efficiency compared to a control editing system not containing one or both of the first and the second MMR polypeptides.

37. (canceled)

38. The method of claim 36, wherein the fold increase is achieved in loci with an editing efficiency lower than 2% when editing using the control editing system.


SEQUENCE LISTING (52 sequences)

SEQ ID NO: 1 - protein, 934 aa, Homo sapiens [sequence omitted]

SEQ ID NO: 2 - protein, 950 aa, synthetic construct
MPAAKRVKLDGGGSVNMAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGKSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 3 - protein, 945 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGKSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTTMLTGPKKKRKV

SEQ ID NO: 4 - protein, 756 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 5 - protein, 772 aa, Homo sapiens
MPAAKRVKLDGGGSVNMSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 6 - protein, 756 aa, synthetic construct; identical to SEQ ID NO: 4

SEQ ID NO: 7 - protein, 1360 aa, Homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 8 - protein, 4128 aa, synthetic construct
ATGCCTGCCGCCAAGAGAGTGAAGCTGGACGGCGGCGGAAGCGTTAACATGTCGCGACAGAGCACCCTGTACAGCTTCTTCCCCAAGTCTCCGGCGCTGAGTGATGCCAACAAGGCCTCGGCCAGGGCCTCACGCGAAGGCGGCCGTGCCGCCGCTGCCCCCGGGGCCTCTCCTTCCCCAGGCGGGGATGCGGCCTGGAGCGAGGCTGGGCCTGGGCCCAGGCCCTTGGCGCGATCCGCGTCACCGCCCAAGGCGAAGAACCTCAACGGAGGGCTGCGGAGATCGGTAGCGCCTGCTGCCCCCACCAGTTGTGACTTCTCACCGGGAGATTTGGTTTGGGCCAAGATGGAGGGTTACCCCTGGTGGCCTTGTCTGGTTTACAACCACCCCTTTGATGGAACATTCATCCGCGAGAAAGGGAAATCAGTCCGTGTTCATGTACAGTTTTTTGATGACAGCCCAACAAGGGGCTGGGTTAGCAAAAGGCTTTTAAAGCCATATACAGGTTCAAAATCAAAGGAAGCCCAGAAGGGAGGTCATTTTTACAGTGCAAAGCCTGAAATACTGAGAGCAATGCAACGTGCAGACGAAGCCTTAAATAAAGACAAGATTAAGAGGCTTGAATTGGCAGTTTGTGATGAGCCCTCAGAGCCAGAAGAGGAAGAAGAGATGGAGGTAGGCACAACTTACGTAACAGATAAGAGTGAAGAAGATAATGAAATTGAGAGTGAAGAGGAAGTACAGCCTAAGACACAAGGATCTAGGCGAAGTAGCCGCCAAATAAAAAAACGAAGGGTCATATCAGATTCTGAGAGTGACATTGGTGGCTCTGATGTGGAATTTAAGCCAGACACTAAGGAGGAAGGAAGCAGTGATGAAATAAGCAGTGGAGTGGGGGATAGTGAGAGTGAAGGCCTGAACAGCCCTGTCAAAGTTGCTCGAAAGCGGAAGAGAATGGTGACTGGAAATGGCTCTCTTAAAAGGAAAAGCTCTAGGAAGGAAACGCCCTCAGCCACCAAACAAGCAACTAGCATTTCATCAGAAACCAAGAATACTTTGAGAGCTTTCTCTGCCCCTCAAAATTCTGAATCCCAAGCCCACGTTAGTGGAGGTGGTGATGACAGTAGTCGCCCTACTGTTTGGTATCATGAAACTTTAGAATGGCTTAAGGAGGAAAAGAGAAGAGATGAGCACAGGAGGAGGCCTGATCACCCCGATTTTGATGCATCTACACTCTATGTGCCTGAGGATTTCCTCAATTCTTGTACTCCTGGGATGAGGAAGTGGTGGCAGATTAAGTCTCAGAACTTTGATCTTGTCATCTGTTACAAGGTGGGGAAATTTTATGAGCTGTACCACATGGATGCTCTTATTGGAGTCAGTGAACTGGGGCTGGTATTCATGAAAGGCAACTGGGCCCATTCTGGCTTTCCTGAAATTGCATTTGGCCGTTATTCAGATTCCCTGGTGCAGAAGGGCTATAAAGTAGCACGAGTGGAACAGACTGAGACTCCAGAAATGATGGAGGCACGATGTAGAAAGATGGCACATATATCCAAGTATGATAGAGTGGTGAGGAGGGAGATCTGTAGGATCATTACCAAGGGTACACAGACTTACAGTGTGCTGGAAGGTGATCCCTCTGAGAACTACAGTAAGTATCTTCTTAGCCTCAAAGAAAAAGAGGAAGATTCTTCTGGCCATACTCGTGCATATGGTGTGTGCTTTGTTGATACTTCACTGGGAAAGTTTTTCATAGGTCAGTTTTCAGATGATCGCCATTGTTCGAGATTTAGGACTCTAGTGGCACACTATCCCCCAGTACAAGTTTTATTTGAAAAAGGAAATCTCTCAAAGGAAACTAAAACAATTCTAAAGAGTTCATTGTCCTGTTCTCTTCAGGAAGGTCTGATACCCGGCTCCCAGTTTTGGGATGCATCCAAAACTTTGAGAACTCTCCTTGAGGAAGAATATTTTAGGGAAAAGCTAAGTGATGGCATTGGGGTGATGTTACCCCAGGTGCTTAAAGGTATGACTTCAGAGTCTGATTCCATTGGGTTGACACCAGGAGAGAAAAGTGAATTGGCCCTCTCTGCTCTAGGTGGTTGTGTCTTCTACCTCAAAAAATGCCTTATTGATCAGGAGCTTTTATCAATGGCTAATTTTGAAGAATATATTCCCTTGGATTCTGACACAGTCAGCACTACAAGATCTGGTGCTATCTTCACCAAAGCCTATCAACGAATGGTGCTAGATGCAGTGACATTAAACAACTTGGAGATTTTTCTGAATGGAACAAATGGTTCTACTGAAGGAACCCTACTAGAGAGGGTTGATACTTGCCATACTCCTTTTGGTAAGCGGCTCCTAAAGCAATGGCTTTGTGCCCCACTCTGTAACCATTATGCTATTAATGATCGTCTAGATGCCATAGAAGACCTCATGGTTGTGCCTGACAAAATCTCCGAAGTTGTAGAGCTTCTAAAGAAGCTTCCAGATCTTGAGAGGCTACTCAGTAAAATTCATAATGTTGGGTCTCCCCTGAAGAGTCAGAACCACCCAGACAGCAGGGCTATAATGTATGAAGAAACTACATACAGCAAGAAGAAGATTATTGATTTTCTTTCTGCTCTGGAAGGATTCAAAGTAATGTGTAAAATTATAGGGATCATGGAAGAAGTTGCTGATGGTTTTAAGTCTAAAATCCTTAAGCAGGTCATCTCTCTGCAGACAAAAAATCCTGAAGGTCGTTTTCCTGATTTGACTGTAGAATTGAACCGATGGGATACAGCCTTTGACCATGAAAAGGCTCGAAAGACTGGACTTATTACTCCCAAAGCAGGCTTTGACTCTGATTATGACCAAGCTCTTGCTGACATAAGAGAAAATGAACAGAGCCTCCTGGAATACCTAGAGAAACAGCGCAACAGAATTGGCTGTAGGACCATAGTCTATTGGGGGATTGGTAGGAACCGTTACCAGCTGGAAATTCCTGAGAATTTCACCACTCGCAATTTGCCAGAAGAATACGAGTTGAAATCTACCAAGAAGGGCTGTAAACGATACTGGACCAAAACTATTGAAAAGAAGTTGGCTAATCTCATAAATGCTGAAGAACGGAGGGATGTATCATTGAAGGACTGCATGCGGCGACTGTTCTATAACTTTGATAAAAATTACAAGGACTGGCAGTCTGCTGTAGAGTGTATCGCAGTGTTGGATGTTTTACTGTGCCTGGCTAACTATAGTCGAGGGGGTGATGGTCCTATGTGTCGCCCAGTAATTCTGTTGCCGGAAGATACCCCCCCCTTCTTAGAGCTTAAAGGATCACGCCATCCTTGCATTACGAAGACTTTTTTTGGAGATGATTTTATTCCTAATGACATTCTAATAGGCTGTGAGGAAGAGGAGCAGGAAAATGGCAAAGCCTATTGTGTGCTTGTTACTGGACCAAATATGGGGGGCAAGTCTACGCTTATGAGACAGGCTGGCTTATTAGCTGTAATGGCCCAGATGGGTTGTTACGTCCCTGCTGAAGTGTGCAGGCTCACACCAATTGATAGAGTGTTTACTAGACTTGGTGCCTCAGACAGAATAATGTCAGGTGAAAGTACATTTTTTGTTGAATTAAGTGAAACTGCCAGCATACTCATGCATGCAACAGCACATTCTCTGGTGCTTGTGGATGAATTAGGAAGAGGTACTGCAACATTTGATGGGACGGCAATAGCAAATGCAGTTGTTAAAGAACTTGCTGAGACTATAAAATGTCGTACATTATTTTCAACTCACTACCATTCATTAGTAGAAGATTATTCTCAAAATGTTGCTGTGCGCCTAGGACATATGGCATGCATGGTAGAAAATGAATGTGAAGACCCCAGCCAGGAGACTATTACGTTCCTCTATAAATTCATTAAGGGAGCTTGTCCTAAAAGCTATGGCTTTAATGCAGCAAGGCTTGCTAATCTCCCAGAGGAAGTTATTCAAAAGGGACATAGAAAAGCAAGAGAATTTGAGAAGATGAATCAGTCACTACGATTATTTCGGGAAGTTTGCCTGGCTAGTGAAAGGTCAACTGTAGATGCTGAAGCTGTCCATAAATTGCTGACTTTGATTAAGGAATTA

SEQ ID NO: 9 - protein, 1371 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKELMLTGPKKKRKV

SEQ ID NO: 10 - protein, 862 aa, synthetic construct
MERAESSSTEPAKAIKPIDRKSVHQICSGQVVLSLSTAVKELVENSLDAGATNIDLKLKDYGVDLIEVSDNGCGVEEENFEGLTLKHHTSKIQEFADLTQVETFGFRGEALSSLCALSDVTISTCHASAKVGTRLMFDHNGKIIQKTPYPRPRGTTVSVQQLFSTLPVRHKEFQRNIKKEYAKMVQVLHAYCIISAGIRVSCTNQLGQGKRQPVVCTGGSPSIKENIGSVFGQKQLQSLIPFVQLPPSDSVCEEYGLSCSDALHNLFYISGFISQCTHGVGRSSTDRQFFFINRRPCDPAKVCRLVNEVYHMYNRHQYPFVVLNISVDSECVDINVTPDKRQILLQEEKLLLAVLKTSLIGMFDSDVNKLNVSQQPLLDVEGNLIKMHAADLEKPMVEKQDQSPSLRTGEEKKDVSISRLREAFSLRHTTENKPHSPKTPEPRRSPLGQKRGMLSSSTSGAISDKGVLRPQKEAVSSSHGPSDPTDRAEVEKDSGHGSTSVDSEGFSIPDTGSHCSSEYAASSPGDRGSQEHVDSQEKAPETDDSFSDVDCHSNQEDTGCKFRVLPQPTNLATPNTKRFKKEEILSSSDICQKLVNTQDMSASQVDVAVKINKKVVPLDFSISSLAKRIKQLHHEAQQSEGEQNYRKFRAKICPGENQAAEDELRKEISKTMFAEMEIIGQFNLGFIITKLNEDIFIVDQHATDEKYNFEMLQQHTVLQGQRLIAPQTLNLTAVNEAVLIENLEIFRKNGFDFVIDENAPVTERAKLISLPTSKNWTFGPQDVDELIFMLSDSPGVMCRPSRVKQMFASRACRKSVMIGTALNTSEMKKLITHMGEMDHPWNCPHGRPTMRHIANLGVISQN

SEQ ID NO: 11 - protein, 862 aa, Homo sapiens; identical to SEQ ID NO: 10

SEQ ID NO: 12 - protein, 873 aa, synthetic construct
MERAESSSTEPAKAIKPIDRKSVHQICSGQVVLSLSTAVKELVENSLDAGATNIDLKLKDYGVDLIEVSDNGCGVEEENFEGLTLKHHTSKIQEFADLTQVETFGFRGEALSSLCALSDVTISTCHASAKVGTRLMFDHNGKIIQKTPYPRPRGTTVSVQQLFSTLPVRHKEFQRNIKKEYAKMVQVLHAYCIISAGIRVSCTNQLGQGKRQPVVCTGGSPSIKENIGSVFGQKQLQSLIPFVQLPPSDSVCEEYGLSCSDALHNLFYISGFISQCTHGVGRSSTDRQFFFINRRPCDPAKVCRLVNEVYHMYNRHQYPFVVLNISVDSECVDINVTPDKRQILLQEEKLLLAVLKTSLIGMFDSDVNKLNVSQQPLLDVEGNLIKMHAADLEKPMVEKQDQSPSLRTGEEKKDVSISRLREAFSLRHTTENKPHSPKTPEPRRSPLGQKRGMLSSSTSGAISDKGVLRPQKEAVSSSHGPSDPTDRAEVEKDSGHGSTSVDSEGFSIPDTGSHCSSEYAASSPGDRGSQEHVDSQEKAPETDDSFSDVDCHSNQEDTGCKFRVLPQPTNLATPNTKRFKKEEILSSSDICQKLVNTQDMSASQVDVAVKINKKVVPLDFSISSLAKRIKQLHHEAQQSEGEQNYRKFRAKICPGENQAAEDELRKEISKTMFAEMEIIGQFNLGFIITKLNEDIFIVDQHATDEKYNFEMLQQHTVLQGQRLIAPQTLNLTAVNEAVLIENLEIFRKNGFDFVIDENAPVTERAKLISLPTSKNWTFGPQDVDELIFMLSDSPGVMCRPSRVKQMFASRACRKSVMIGTALNTSEMKKLITHMGEMDHPWNCPHGRPTMRHIANLGVISQNMLTGPKKKRKV

SEQ ID NO: 13 - protein, 934 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGRSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 14 - protein, 950 aa, synthetic construct
MPAAKRVKLDGGGSVNMAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGRSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 15 - protein, 945 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGRSTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTTMLTGPKKKRKV

SEQ ID NO: 16 - protein, 934 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGASTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 17 - protein, 950 aa, synthetic construct
MPAAKRVKLDGGGSVNMAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGASTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 18 - protein, 945 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGASTYIRQTGVIVLMAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTTMLTGPKKKRKV

SEQ ID NO: 19 - protein, 934 aa, synthetic construct [sequence omitted]

SEQ ID NO: 20 - protein, 950 aa, synthetic construct
MPAAKRVKLDGGGSVNMAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGKSTYIRQTGVIVLRAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTT

SEQ ID NO: 21 - protein, 945 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVRPAILEKGQGRIILKASRHACVEVQDEIAFIPNDVYFEKDKQMFHIITGPNMGGKSTYIRQTGVIVLRAQIGCFVPCESAEVSIVDCILARVGAGDSQLKGVSTFMAEMLETASILRSATKDSLIIIDELGRGTSTYDGFGLAWAISEYIATKIGAFCMFATHFHELTALANQIPTVNNLHVTALTTEETLTMLYQVKKGVCDQSFGIHVAELANFPKHVIECAKQKALELEEFQYIGESQGYDIMEPAAKKCYLEREQGEKIIQEFLSKVKQMPFTEMSEENITIKLKQLKAEVIAKNNSFVNEIISRIKVTTMLTGPKKKRKV

SEQ ID NO: 22 - protein, 620 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYV

SEQ ID NO: 23 - protein, 636 aa, synthetic construct
MPAAKRVKLDGGGSVNMAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYV

SEQ ID NO: 24 - protein, 631 aa, synthetic construct
MAVQPKETLQLESAAEVGFVRFFQGMPEKPTTTVRLFDRGDFYTAHGEDALLAAREVFKTQGVIKYMGPAGAKNLQSVVLSKMNFESFVKDLLLVRQYRVEVYKNRAGNKASKENDWYLAYKASPGNLSQFEDILFGNNDMSASIGVVGVKMSAVDGQRQVGVGYVDSIQRKLGLCEFPDNDQFSNLEALLIQIGPKECVLPGGETAGDMGKLRQIIQRGGILITERKKADFSTKDIYQDLNRLLKGKKGEQMNSAVLPEMENQVAVSSLSAVIKFLELLSDDSNFGQFELTTFDFSQYMKLDIAAVRALNLFQGSVEDTTGSQSLAALLNKCKTPQGQRLVNQWIKQPLMDKNRIEERLNLVEAFVEDAELRQTLQEDLLRRFPDLNRLAKKFQRQAANLQDCYRLYQGINQLPNVIQALEKHEGKHQKLLLAVFVTPLTDLRSDFSKFQEMIETTLDMDQVENHEFLVKPSFDPNLSELREIMNDLEKKMQSTLISAARDLGLDPGKQIKLDSSAQFGYYFRVTCKEEKVLRNNKNFSTVDIQKNGVKFTNSKLTSLNEEYTKNKTEYEEAQDAIVKEIVNISSGYVEPMQTLNDVLAQLDAVVSFAHVSNGAPVPYVMLTGPKKKRKV

SEQ ID NO: 25 - protein, 337 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGS

SEQ ID NO: 26 - protein, 353 aa, synthetic construct
MPAAKRVKLDGGGSVNMSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGS

SEQ ID NO: 27 - protein, 348 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSMLTGPKKKRKV

SEQ ID NO: 28 - protein, 1360 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKAYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 29 - protein, 1376 aa, synthetic construct
MPAAKRVKLDGGGSVNMSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKAYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 30 - protein, 1371 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKAYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKELMLTGPKKKRKV

SEQ ID NO: 31 - protein, 1360 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGRSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 32 - protein, 1376 aa, synthetic construct
MPAAKRVKLDGGGSVNMSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGRSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 33 - protein, 1371 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGRSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKELMLTGPKKKRKV

SEQ ID NO: 34 - protein, 1360 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVRAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 35 - protein, 1376 aa, synthetic construct
MPAAKRVKLDGGGSVNMSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVRAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 36 - protein, 1371 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVRAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKELMLTGPKKKRKV

SEQ ID NO: 37 - protein, 1075 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMC

SEQ ID NO: 38 - protein, 1091 aa, synthetic construct
MPAAKRVKLDGGGSVNMSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMC

SEQ ID NO: 39 - protein, 1086 aa, synthetic construct
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCMLTGPKKKRKV

SEQ ID NO: 40 - protein, 499 aa, synthetic construct
MSDVNKLNVSQQPLLDVEGNLIKMHAADLEKPMVEKQDQSPSLRTGEEKKDVSISRLREAFSLRHTTENKPHSPKTPEPRRSPLGQKRGMLSSSTSGAISDKGVLRPQKEAVSSSHGPSDPTDRAEVEKDSGHGSTSVDSEGFSIPDTGSHCSSEYAASSPGDRGSQEHVDSQEKAPETDDSFSDVDCHSNQEDTGCKFRVLPQPTNLATPNTKRFKKEEILSSSDICQKLVNTQDMSASQVDVAVKINKKVVPLDFSISSLAKRIKQLHHEAQQSEGEQNYRKFRAKICPGENQAAEDELRKEISKTMFAEMEIIGQFNLGFIITKLNEDIFIVDQHATDEKYNFEMLQQHTVLQGQRLIAPQTLNLTAVNEAVLIENLEIFRKNGFDFVIDENAPVTERAKLISLPTSKNWTFGPQDVDELIFMLSDSPGVMCRPSRVKQMFASRACRKSVMIGTALNTSEMKKLITHMGEMDHPWNCPHGRPTMRHIANLGVISQN

SEQ ID NO: 41 - skipped

SEQ ID NO: 42 - protein, 532 aa, synthetic construct
MSDVNKLNVSQQPLLDVEGNLIKMHAADLEKPMVEKQDQSPSLRTGEEKKDVSISRLREAFSLRHTTENKPHSPKTPEPRRSPLGQKRGMLSSSTSGAISDKGVLRPQKEAVSSSHGPSDPTDRAEVEKDSGHGSTSVDSEGFSIPDTGSHCSSEYAASSPGDRGSQEHVDSQEKAPETDDSFSDVDCHSNQEDTGCKFRVLPQPTNLATPNTKRFKKEEILSSSDICQKLVNTQDMSASQVDVAVKINKKVVPLDFSISSLAKRIKQLHHEAQQSEGEQNYRKFRAKICPGENQAAEDELRKEISKTMFAEMEIIGQFNLGFIITKLNEDIFIVDQHATDEKYNFEMLQQHTVLQGQRLIAPQTLNLTAVNEAVLIENLEIFRKNGFDFVIDENAPVTERAKLISLPTSKNWTFGPQDVDELIFMLSDSPGVMCRPSRVKQMFASRACRKSVMIGTALNTSEMKKLITHMGEMDHPWNCPHGRPTMRHIANLGVISQNATGTTAACTGGCCCAAAGAAGAAGCGGAAAGTC

SEQ ID NO: 43 - protein, 337 aa, synthetic construct; identical to SEQ ID NO: 25

SEQ ID NO: 44 - protein, 353 aa, synthetic construct; identical to SEQ ID NO: 26

SEQ ID NO: 45 - protein, 348 aa, synthetic construct; identical to SEQ ID NO: 27

SEQ ID NO: 46 - protein, 753 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 47 - protein, 769 aa, synthetic construct
MPAAKRVKLDGGGSVNMSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 48 - protein, 764 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFMLTGPKKKRKV

SEQ ID NO: 49 - skipped

SEQ ID NO: 50 - protein, 1157 aa, Homo sapiens
MIKCLSVEVQAKLRSGLAISSLGQCVEELALNSIDAEAKCVAVRVNMETFQVQVIDNGFGMGSDDVEKVGNRYFTSKCHSVQDLENPRFYGFRGEALANIADMASAVEISSKKNRTMKTFVKLFQSGKALKACEADVTRASAGTTVTVYNLFYQLPVRRKCMDPRLEFEKVRQRIEALSLMHPSISFSLRNDVSGSMVLQLPKTKDVCSRFCQIYGLGKSQKLREISFKYKEFELSGYISSEAHYNKNMQFLFVNKRLVLRTKLHKLIDFLLRKESIICKPKNGPTSRQMNSSLRHRSTPELYGIYVINVQCQFCEYDVCMEPAKTLIEFQNWDTLLFCIQEGVKMFLKQEKLFVELSGEDIKEFSEDNGFSLFDATLQKRVTSDERSNFQEACNNILDSYEMFNLQSKAVKRKTTAENVNTQSSRDSEATRKNTNDAFLYIYESGGPGHSKMTEPSLQNKDSSCSESKMLEQETIVASEAGENEKHKKSFLEHSSLENPCGTSLEMFLSPFQTPCHFEESGQDLEIWKESTTVNGMAANILKNNRIQNQPKRFKDATEVGCQPLPFATTLWGVHSAQTEKEKKKESSNCGRRNVFSYGRVKLCSTGFITHVVQNEKTKSTETEHSFKNYVRPGPTRAQETFGNRTRHSVETPDIKDLASTLSKESGQLPNKKNCRTNISYGLENEPTATYTMFSAFQEGSKKSQTDCILSDTSPSFPWYRHVSNDSRKTDKLIGFSKPIVRKKLSLSSQLGSLEKFKRQYGKVENPLDTEVEESNGVTTNLSLQVEPDILLKDKNRLENSDVCKITTMEHSDSDSSCQPASHILNSEKFPFSKDEDCLEQQMPSLRESPMTLKELSLFNRKPLDLEKSSESLASKLSRLKGSERETQTMGMMSRFNELPNSDSSRKDSKLCSVLTQDFCMLFNNKHEKTENGVIPTSDSATQDNSFNKNSKTHSNSNTTENCVISETPLVLPYNNSKVTGKDSDVLIRASEQQIGSLDSPSGMLMNPVEDATGDQNGICFQSEESKARACSETEESNTCCSDWQRHFDVALGRMVYVNKMTGLSTFIAPTEDIQAACTKDLTTVAVDVVLENGSQYRCQPFRSDLVLPFLPRARAERTVMRQDNRDTVDDTVSSESLQSLFSEWDNPVFARYPESF

SEQ ID NO: 51 - protein, 1137 aa, Homo sapiens
MSRRKPASGGLAASSSAPARQAVLSRFFQSTGSLKSTSSSTGAADQVDPGAAAAAAAAAAAAPPAPPAPAFPPQLPPHIATEIDRRKKRPLENDGPVKKKVKKVQQKEGGSDLGMSGNSEPKKCLRTRNVSKSLEKLKEFCCDSALPQSRVQTESLQERFAVLPKCTDFDDISLLHAKNAVSSEDSKRQINQKDTTLFDLSQFGSSNTSHENLQKTASKSANKRSKSIYTPLELQYIEMKQQHKDAVLCVECGYKYRFFGEDAEIAARELNIYCHLDHNFMTASIPTHRLFVHVRRLVAKGYKVGVVKQTETAALKAIGDNRSSLFSRKLTALYTKSTLIGEDVNPLIKLDDAVNVDEIMTDTSTSYLLCISENKENVRDKKKGNIFIGIVGVQPATGEVVFDSFQDSASRSELETRMSSLQPVELLLPSALSEQTEALIHRATSVSVQDDRIRVERMDNIYFEYSHAFQAVTEFYAKDTVDIKGSQIISGIVNLEKPVICSLAAIIKYLKEFNLEKMLSKPENFKQLSSKMEFMTINGTTLRNLEILQNQTDMKTKGSLLWVLDHTKTSFGRRKLKKWVTQPLLKLREINARLDAVSEVLHSESSVFGQIENHLRKLPDIERGLCSIYHKKCSTQEFFLIVKTLYHLKSEFQAIIPAVNSHIQSDLLRTVILEIPELLSPVEHYLKILNEQAAKVGDKTELFKDLSDFPLIKKRKDEIQGVIDEIRMHLQEIRKILKNPSAQYVTVSGQEFMIEIKNSAVSCIPTDWVKVGSTKAVSRFHSPFIVENYRHLNQLREQLVLDCSAEWLDFLEKFSEHYHSLCKAVHHLATVDCIFSLAKVAKQGDYCRPTVQEERKIVIKNGRHPVIDVLLGEQDQYVPNNTDLSEDSERVMIITGPNMGGKSSYIKQVALITIMAQIGSYVPAEEATIGIVDGIFTRMGAADNIYKGQSTFMEELTDTAEIIRKATSQSLVILDELGRGTSTHDGIAIAYATLEYFIRDVKSLTLFVTHYPPVCELEKNYSHQVGNYHMGFLVSEDESKLDPGAAEQVPDFVTFLYQITRGIAARSYGLNVAKLADVPGEILKKAAHKSKELEGLINTKRKRLKYFAKLWTMHNAQDLQKWTEEFNMEETQTSLLH

SEQ ID NO: 52 - protein, 709 aa, Homo sapiens
MKQLPAATVRLLSSSQIITSVVSVVKELIENSLDAGATSVDVKLVLITTRTAADNFSTQYVLDGSGHILSQKPSHLGQGTTVTALRLFKNLPVRKQFYSTAKKCKDEIKKIQDLLMSFGILKPDLRIVFVHNKAVIWQKSRVSDHKMALMSVLGTAVMNNMESFQYHSEESQIYLSGFLPKCDADHSFTSLSTPERSFIFINSRPVHQKDILKLIRHHYNLKCLKESTRLYPVFFLKIDVPTADVDVNLTPDKSQVLLQNKESVLIALENLMTTCYGPLPSTNSYENNKTDVSAADIVLSKTAETDVLFNKVESSGKNYSNVDTSVIPFQNDMHNDESGKNTDDCLNHQISIGDFGYGHCSSEISNIDKNTKNAFQDISMSNVSWENSQTEYSKTCFISSVKHTQSENGNKDHIDESGENEEEAGLENSSEISADEWSRGNILKNSVGENIEPVKILVPEKSLPCKVSNNNYPIPEQMNLNEDSCNKKSNVIDNKSGKVTAYDLLSNRVIKKPMSASALFVQDHRPQFLIENPKTSLEDATLQIEELWKTLSEEEKLNLFNGSHYLDVLYKMTADDQRYSGSTYLSDPRLTANGFKIKLIPGVSITENYLEIEGMANCLPFYGVADLKEILNAILNRNAKEVYECRPRKVISYLEGEAVRLSRQLPMYLSKEDIQDIIYRMKHQFGNEIKECVHGRPFFHHLTYLPETT